SEQUENCE LISTING (24 sequences)

SEQ ID NO: 1 - DNA, 2186 bp, Homo sapiens
  CDS (20)..(2011)
  misc_feature (2150)..(2150): wherein “n” equals A, C, G, or T.
ctctagatgtacatggaggatgaccgaaaaaaccaatggtgtgaagagctccccagccaataatcacaaccatcatgcacctcctgccatcaaggccaatggcaaagatgaccacaggacaagcagcaggccacactctgcagctgacgatgacacctcctcagaactgcagaggctggcagacgtggatgccccacagcagggaaggagtggcttccgcaggatagttcgcctggtggggatcatcagagaatgggccaacaagaatttccgagaggaggaacctaggcctgactcattcctcgagcgttttcgtgggcctgaactccagactgtgaccacacaggagggggatggcaaaggcgacaaggatggcgaggacaaaggcaccaagaagaaatttgaactatttgtcttggacccagctggggatttgtactactgctggctatttgtcattgccatgcccgtcctttacaactggtgcctgctggtggccagagcctgcttcagtgacctacagaaaggctactacctggtgtggctggtgctggattatgtctcagatgtggtctacattgcggacctcttcatccgattgcgcacaggtttcctggagcaggggctgctggtcaaagataccaagaaactgcgagacaactacatccacaccctgcagttcaagctggatgtggcttccatcatccccactgacctgatctattttgctgtggacatccacagccctgaggtgcgcttcaaccgcctgctgcactttgcccgcatgtttgagttctttgaccggacagagacacgcaccaactaccctaacatcttccgcatcagcaaccttgtcctctacatcttggtcatcatccactggaatgcctgcatctattatgccatctccaaatccataggctttggggtcgacacctgggtttacccaaacatcactgaccctgagtatggctacctggctagggaatacatctattgcctttactggtccacactgactctcactaccattggggagacaccaccccctgtaaaggatgaggagtacctatttgtcatctttgacttcctgattggcgtcctcatctttgccaccatcgtgggaaatgtgggctccatgatctccaacatgaatgccacccgggcagagttccaggctaagatcgatgccgtgaaacactacatgcagttccgaaaggtcagcaaggggatggaagccaaggtcattaggtggtttgactacttgtggaccaataagaagacagtggatgagcgagaaattctcaagaatctgccagccaagctcagggctgagatagccaccaatgtccacttgtccacactcaagaaagtgcgcatcttccatgattgtgaggctggcctgctggtagagctggtactgaaactccgtcctcaggtcttcagtcctggggattacatttgccgcaaaggggacatcggcaaggagatgtacatcattaaggagggcaaactggcagtggtggctgatgatggtgtgactcagtatgctctgctgtcggctggaagctgctttggcgagatcagtatccttaacattaagggcagtaaaatgggcaatcgacgcacagctaatatccgcagcctgggctactcagatctcttctgcttgtccaaggatgatcttatggaagctgtgactgagtaccctgatgccaagaaagtcctagaagagaggggtcgggagatcctcatgaaggagggactgctggatgagaacgaagtggcaaccagcatggaggtcgacgtgcaggagaagctagggcagctggagaccaacatggaaaccttgtacactcgctttggccgcctgctggctgagtacacgggggcccagcagaagctcaagcagcgcatcacagttctggaaaccaagatgaaacagaacaatgaagatgactacctgtctgatgggatgaacagccctgagctggctgctgctgacgagccataagacctggggcccaactgcctctccagcattggccttggccttgatcccagaagctagaggagctatttagatctccggatttacatgcattaccctcatgttccctgaattctcccaaaagtctctctgaccctgngtttttggcctaaacatccaagattccgcctcggat

SEQ ID NO: 2 - protein, 664 aa, Homo sapiens
MTEKTNGVKSSPANNHNHHAPPAIKANGKDDHRTSSRPHSAADDDTSSELQRLADVDAPQQGRSGFRRIVRLVGIIREWANKNFREEEPRPDSFLERFRGPELQTVTTQEGDGKGDKDGEDKGTKKKFELFVLDPAGDLYYCWLFVIAMPVLYNWCLLVARACFSDLQKGYYLVWLVLDYVSDVVYIADLFIRLRTGFLEQGLLVKDTKKLRDNYIHTLQFKLDVASIIPTDLIYFAVDIHSPEVRFNRLLHFARMFEFFDRTETRTNYPNIFRISNLVLYILVIIHWNACIYYAISKSIGFGVDTWVYPNITDPEYGYLAREYIYCLYWSTLTLTTIGETPPPVKDEEYLFVIFDFLIGVLIFATIVGNVGSMISNMNATRAEFQAKIDAVKHYMQFRKVSKGMEAKVIRWFDYLWTNKKTVDEREILKNLPAKLRAEIATNVHLSTLKKVRIFHDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLDENEVATSMEVDVQEKLGQLETNMETLYTRFGRLLAEYTGAQQKLKQRITVLETKMKQNNEDDYLSDGMNSPELAAADEP

SEQ ID NO: 3 - DNA, 30 bp, Artificial Sequence
  Primer
gctctagatgtacatggaggatgaccgaaa

SEQ ID NO: 4 - DNA, 22 bp, Artificial Sequence
  Primer
cagccaacgcagtctgtactct

SEQ ID NO: 5 - DNA, 29 bp, Artificial Sequence
  Primer
cgggatccgaggcggaatcttggatgttt

SEQ ID NO: 6 - DNA, 17 bp, Artificial Sequence
  Primer
agagcctgcttcagtga

SEQ ID NO: 7 - DNA, 17 bp, Artificial Sequence
  Primer
tcactgaagcaggctct

SEQ ID NO: 8 - DNA, 17 bp, Artificial Sequence
  Primer
ttactggtccacactga

SEQ ID NO: 9 - DNA, 17 bp, Artificial Sequence
  Primer
tcagtgtggaccagtaa

SEQ ID NO: 10 - DNA, 20 bp, Artificial Sequence
  Primer
acgcacagctaatatccgca

SEQ ID NO: 11 - DNA, 20 bp, Artificial Sequence
  Primer
tgcggatattagctgtgcgt

SEQ ID NO: 12 - DNA, 21 bp, Artificial Sequence
  Primer
tcagagaatgggccaacaaga

SEQ ID NO: 13 - DNA, 20 bp, Artificial Sequence
  Primer
cgaaaacgctcgaggaatga

SEQ ID NO: 14 - DNA, 26 bp, Artificial Sequence
  Primer/Probe
caggcctaggttcctcctctcggaaa

SEQ ID NO: 15 - protein, 732 aa, Oryctolagus cuniculus
MSSWRSCARAPLSGSAWRRSAATRRSRRCLKTKRKRWSSGKGTPMQSTQCETRRRAQTPCESTGHTWRMTEKSNGVKSSPANNHNNHVPATIKANGKDESRTRSRPQSAADDDTSSELQRLAEMDAPQQRRGGFRRIVRLVGVIRQWANRNFREEEARPDSFLERFRGPELQTVTTQQGDGKGDKDGDGKGTKKKFELFVLDPAGDWYYRWLFVIAMPVLYNWCLLVARACFSDLQRGYFLVWLVLDYFSDVVYIADLFIRLRTGFLEQGLLVKDPKKLRDNYIHTLQFKLDVASIIPTDLIYFAVGIHNPELRFNRLLHFARMFEFFDRTETRTSYPNIFRISNLVLYILVIIHWNACIYYAISKSIGFGVDTWVYPNITDPEYGYLAREYIYCLYWSTLTLTTIGETPPPVKDEEYLFVIFDFLIGVLIFATIVGNVGSMISNMNATRAEFQAKIDAVKHYMQFRKVSKEMEAKVIKWFDYLWTNKKTVDEREVLKNLPAKLRAEIAINVHLSTLKKVRIFQDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLDENEVAASMEVDVQEKLKQLETNMETLYTRFGRLLAEYTGAQQKLKQRITVLEVKMKQNTEDDYLSDGMNSPEPAAAEQP

SEQ ID NO: 16 - protein, 663 aa, Bos taurus
MTEKANGVKSSPANNHNHHAPPAIKASGKDDHRASSRPQSAAADDTSSELQQLAEMDAPQQRRGGFRRIARLVGVLREWAYRNFREEEPRPDSFLERFRGPELHTVTTQQGDGKGDKDGEGKGTKKKFELFVLDPAGDWYYRWLFLIALPVLYNWCLLVARACFSDLQKGYYIVWLVLDYVSDVVYIADLFIRLRTGFLEQGLLVKDTKKLRDNYIHTMQFKLDVASIIPTDLIYFAVGIHNPEVRFNRLLHFARMFEFFDRTETRTSYPNIFRISNLILYILIIIHWNACIYYAISKSIGFGVDTWVYPNITDPEYGYLSREYIYCLYWSTLTLTTIGETPPPVKDEEYLFVIFDFLIGVLIFATIVGNVGSMISNMNATRAEFQAKIDAVKHYMQFRKVSKEMEAKVIRWFDYLWTNKKSVDEREVLKNLPAKLRAEIAINVHLSTLKKVRIFQDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKRVLEERGREILMKEGLLDENEVAASMEVDVQEKLEQLETNMDTLYTRFARLLAEYTGAQQKLKQRITVLETKMKQNNEDDSLSDGMNSPEPPAEKP

SEQ ID NO: 17 - protein, 664 aa, Mus musculus
MMTEKSNGVKSSPANNHNHHPPPSIKANGKDDHRAGSRPQSVAADDDTSSELQRLAEMDTPRRGRGGFRRIVRLVGIIRDWANKNFREEEPRPDSFLERFRGPELQTVTPHQGDGKGDKDGEGKGTKKKFELFVLDPAGDWYYRWLFVIAMPVLYNWCLLVARACFSDLQRNYFVVWLVLDYFSDTVYIADLIIRLRTGFLEQGLLVKDPKKLRDNYIHTLQFKLDVASIIPTDLIYFAVGIHSPEVRFNRLLHFARMFEFFDRTETRTSYPNIFRISNLVLYILVIIHWNACIYYAISKSIGFGVDTWVYPNITDPEYGYLAREYIYCLYWSTLTLTTIGETPPPVKDEEYLFFIFDFLIGVLIFATIVGNVGSMISNMNATRAEFQAKIDAVKHYMQFRKVSKDMEAKVIKWFDYLWTNKKTVDEREVLKNLPAKLRAEIAINVHLSTLKKVRIFQDCEAGLLVELVLKLRPQVFSPGDYICRKGDIGKEMYIIKEGKLAVVADDGVTQYALLSAGSCFGEISILNIKGSKMGNRRTGTIRSLGYSDLFCLSKDDLMEAVTEYPDAKKVLEERGREILMKEGLLDENEVAASMEVDVQEKLEQLETNMETLYTRFARLLAEYTGAQQKLKQRITVLETKMKQNHEDDYLSDGINTPEPAVAE

SEQ ID NO: 18 - protein, 664 aa, Rattus norvegicus
MMTEKSNGVKSSPANNHNHHPPPSIKANGKDDHRAGSRPQSVAADDDTSPELQRLAEMDTPRRGRGGFQRIVRLVGVIRDWANKNFREEEPRPDSFLERFRGPELQTVTTHQGDDKGGKDGEGKGTKKKFELFVLDPAGDWYYRWLFVIAMPVLYNWCLLVARACFSDLQRNYFVVWLVLDYFSDTVYIADLIIRLRTGFLEQGLLVKDPKKLRDNYIHTLQFKLDVASIIPTDLIYFAVGIHSPEVRFNRLLHFARMFEFFDRTETRTSYPNIFRISNLVLYILVIIHWNACIYYVISKSIGFGVDTWVYPNITDPEYGYLAREYIYCLYWSTLTLTTIGETPPPVKDEEYLFVIFDFLIGVLIFATIVGNVGSMISNMNATRAEFQAKIDAVKHYMQFRKVSKDMEAKVIKWFDYLWTNKKTVDEREVLKNLPAKLRAEIAINVHLSTLKKVRIFQDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLDENEVAASMEVDVQEKLEQLETNMDTLYTRFARLLAEYTGAQQKLKQRITVLETKMKQNHEDDYLSDGINTPEPTAAE

SEQ ID NO: 19 - DNA, 39 bp, Homo sapiens
gcagcagcggccgctactactgctggctatttgtcattg

SEQ ID NO: 20 - DNA, 36 bp, Homo sapiens
gcagcagtcgactggctcgtcagcagcagccagctc

SEQ ID NO: 21 - DNA, 38 bp, Homo sapiens
gcagcagcggccgcatgaccgaaaaaaccaatggtgtg

SEQ ID NO: 22 - DNA, 36 bp, Homo sapiens
gcagcagtcgacgaagacctgaggacggagtttcag

SEQ ID NO: 23 - DNA, 2190 bp, Homo sapiens
  CDS (20)..(2011)
ctctagatgtacatggaggatgaccgaaaaaaccaatggtgtgaagagctccccagccaataatcacaaccatcatgcacctcctgccatcaaggccaatggcaaagatgaccacaggacaagcagcaggccacactctgcagctgacgatgacacctcctcagaactgcagaggctggcagacgtggatgccccacagcagggaaggagtggcttccgcaggatagttcgcctggtggggatcatcagagaatgggccaacaagaatttccgagaggaggaacctaggcctgactcattcctcgagcgttttcgtgggcctgaactccagactgtgaccacacaggagggggatggcaaaggcgacaaggatggcgaggacaaaggcaccaagaagaaatttgaactatttgtcttggacccagctggggatttgtactactgctggctatttgtcattgccatgcccgtcctttacaactggtgcctgctggtggccagagcctgcttcagtgacctacagaaaggctactacctggtgtggctggtgctggattatgtctcagatgtggtctacattgcggacctcttcatccgattgcgcacaggtttcctggagcaggggctgctggtcaaagataccaagaaactgcgagacaactacatccacaccctgcagttcaagctggatgtggcttccatcatccccactgacctgatctattttgctgtggacatccacagccctgaggtgcgcttcaaccgcctgctgcactttgcccgcatgtttgagttctttgaccggacagagacacgcaccaactaccctaacatcttccgcatcagcaaccttgtcctctacatcttggtcatcatccactggaatgcctgcatctattatgccatctccaaatccataggctttggggtcgacacctgggtttacccaaacatcactgaccctgagtatggctacctggctagggaatacatctattgcctttactggtccacactgactctcactaccattggggagacaccaccccctgtaaaggatgaggagtacctatttgtcatctttgacttcctgattggcgtcctcatctttgccaccatcgtgggaaatgtgggctccatgatctccaacatgaatgccacccgggcagagttccaggctaagatcgatgccgtgaaacactacatgcagttccgaaaggtcagcaaggggatggaagccaaggtcattaggtggtttgactacttgtggaccaataagaagacagtggatgagcgagaaattctcaagaatctgccagccaagctcagggctgagatagccatcaatgtccacttgtccacactcaagaaagtgcgcatcttccatgattgtgaggctggcctgctggtagagctggtactgaaactccgtcctcaggtcttcagtcctggggattacatttgccgcaaaggggacatcggcaaggagatgtacatcattaaggagggcaaactggcagtggtggctgatgatggtgtgactcagtatgctctgctgtcggctggaagctgctttggcgagatcagtatccttaacattaagggcagtaaaatgggcaatcgacgcacagctaatatccgcagcctgggctactcagatctcttctgcttgtccaaggatgatcttatggaagctgtgactgagtaccctgatgccaagaaagtcctagaagagaggggtcgggagatcctcatgaaggagggactgctggatgagaacgaagtggcaaccagcatggaggtcgacgtgcaggagaagctagggcagctggagaccaacatggaaaccttgtacactcgctttggccgcctgctggctgagtacacgggggcccagcagaagctcaagcagcgcatcacagttctggaaaccaagatgaaacagaacaatgaagatgactacctgtctgatgggatgaacagccctgagctggctgctgctgacgagccataagacctggggcccaactgcctctccagcattggccttggccttgatcccagaagctagaggagctatttagatctccggatttacatgcattaccctcatgttccctgaattctcccaaaagcctctctgaccctgggtttttggcctaaacatccaagattccgcctcggatcccg

SEQ ID NO: 24 - protein, 664 aa, Homo sapiens
MTEKTNGVKSSPANNHNHHAPPAIKANGKDDHRTSSRPHSAADDDTSSELQRLADVDAPQQGRSGFRRIVRLVGIIREWANKNFREEEPRPDSFLERFRGPELQTVTTQEGDGKGDKDGEDKGTKKKFELFVLDPAGDLYYCWLFVIAMPVLYNWCLLVARACFSDLQKGYYLVWLVLDYVSDVVYIADLFIRLRTGFLEQGLLVKDTKKLRDNYIHTLQFKLDVASIIPTDLIYFAVDIHSPEVRFNRLLHFARMFEFFDRTETRTNYPNIFRISNLVLYILVIIHWNACIYYAISKSIGFGVDTWVYPNITDPEYGYLAREYIYCLYWSTLTLTTIGETPPPVKDEEYLFVIFDFLIGVLIFATIVGNVGSMISNMNATRAEFQAKIDAVKHYMQFRKVSKGMEAKVIRWFDYLWTNKKTVDEREILKNLPAKLRAEIAINVHLSTLKKVRIFHDCEAGLLVELVLKLRPQVFSPGDYICRKGDIGKEMYIIKEGKLAVVADDGVTQYALLSAGSCFGEISILNIKGSKMGNRRTANIRSLGYSDLFCLSKDDLMEAVTEYPDAKKVLEERGREILMKEGLLDENEVATSMEVDVQEKLGQLETNMETLYTRFGRLLAEYTGAQQKLKQRITVLETKMKQNNEDDYLSDGMNSPELAAADEP

(19) United States
(12) Patent Application Publication
(10) Pub. No.: US 2003/0096249 A1
(43) Pub. Date: May 22, 2003

(54) Nucleic acid molecules and polypeptides for a human cation channel polypeptide

(76) Inventors: Ryan S. Westphal, Cheshire, CT (US); John N. Feder, Belle Mead, NJ (US); Chandra S. Ramanathan, Wallingford, CT (US); Gabriel A. Mintier, Hightstown, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/029,677

(22) Filed: Dec. 21, 2001

Related U.S. Application Data
(60) Provisional application No. 60/257,865, filed on Dec. 21, 2000.

(51) Int. Cl. C12Q 1/68; C07H 21/04; C12P 21/02; C12N 5/06; C07K 14/435

(52) U.S. Cl. 435/6; 435/69.1; 435/320.1; 435/325; 530/350; 536/23.5

(57) ABSTRACT

The present invention relates to novel human nucleic acid molecules encoding novel human cation channels, and proteins and polypeptides encoded by such nucleic acid molecules. More specifically, the nucleic acid molecules of the invention include the novel human gene designated HBMYCNG. The proteins and polypeptides of the invention represent a novel cation channel that may be therapeutically valuable targets for drug delivery in the treatment of human diseases which involve calcium, sodium, potassium or other ionic homeostatic dysfunction, such as central nervous system (CNS) disorders, e.g., stroke, anxiety and depression, or degenerative neurological disorders such as Alzheimer's disease or Parkinson's disease, or other disorders such as cardiac disorders, e.g., arrhythmia, diabetes, chronic pain, hypercalcemia, hypocalcemia, hypercalciuria, hypocalciuria, or ion disorders associated with immunological disorders, gasto-intestinal (GI) tract disorders, or renal or liver disease.

This application claims benefit to provisional application U.S. Serial No. 60/257,865, filed Dec. 21, 2000.

### 1. FIELD OF THE INVENTION

The present invention relates to the isolation and identification of human nucleic acid molecules and proteins and polypeptides encoded by such nucleic acid molecules, or degenerate variants thereof, encoding a human cyclic nucleotide gated (CNG) cation channel. The proteins and polypeptides of the invention represent a novel cation channel that may be a therapeutically valuable target for drug delivery in the treatment of human diseases that involve calcium, sodium, potassium or other ionic homeostatic dysfunction, such as central nervous system (CNS) disorders, e.g., stroke, anxiety and depression, or degenerative neurological disorders such as Alzheimer's disease or Parkinson's disease, or other disorders such as cardiac disorders, e.g., arrhythmia, diabetes, chronic pain, hypercalcemia, hypocalcemia, hypercalciuria, hypocalciuria, or ion disorders associated with immunological disorders, gastrointestinal (GI) tract disorders, or renal or liver disease. Moreover, the polypeptides of the present invention can function as effector molecules, reflecting the intracellular concentration of cAMP and/or cGMP. Accordingly the present invention also relates to the use of the CNG cation channel polypeptides disclosed herein for the detection of modulators of intracellular cAMP and/or cGMP levels. More specifically, the present invention relates to the use of CNG cation channel polypeptides as components of assays for the detection of antagonists and/or agonists of G-protein coupled receptor activity, which may be therapeutically useful molecules.

### 2. BACKGROUND OF THE INVENTION

Control of the internal ionic environment is an extremely important function of all living cells. Ion exchange with the external medium is regulated by a variety of means, the most important of which are various transporters and ion channels. Ion channels in particular have been important targets for the development of therapeutic compounds in the treatment of disease.

A number of proteins have been described as forming ion channels. Among these are proteins that have been shown to function as cation channels of varying degrees of selectivity and with different, and in some cases unknown, mechanisms for channel gating. Within the family of cation channels, there is an identified group that includes cyclic nucleotide gated (CNG) channels, which are activated by intracellular binding of cAMP and/or cGMP to CNG polypeptides. CNG channels are nonselective cation channels which allow the passage of monovalent cations, including both K⁺ and Na⁺ ions, as well as divalent cations. Although CNG channels can transport both monovalent and divalent cations, Ca⁺² blocks the flow of monovalent cations through the channel (Zagotta et al. 1996 Ann. Rev. Neurosci. 19: 235-63). CNG channels were originally found to be involved in signal transduction within sensory tissues.

The first cDNA clone encoding a CNG channel α-subunit polypeptide was isolated from bovine rod tissue (Kaupp et al. 1989 Nature 342: 762-66). Subsequently, a series of CNG α-subunit polypeptide encoding genes were isolated from other tissues and species that encoded proteins structurally related to the bovine rod CNG α-subunit polypeptide. (Bauman et al. 1994 EMBO J 13:5040-50; Biel et al. 1993 FEBS Lett 329: 134-38; Biel et al. 1994 Proc. Natl. Acad. Sci. USA 91:3505-09; Bönigk et al. 1993 Neuron 10: 865-77; Bradley et al. 1994 Proc. Natl. Acad. Sci. USA 91: 8890-94; Chen et al. 1993 Nature 362: 764-67; Dhallan et al. 1990 Nature 347: 184-87; Dhallan et al. 1992 J. Neurosci. 12:3248-56; Goulding et al. 1992 Neuron 8: 45-58; Liman et al. 1994 Neuron 13: 611-21; Ludwig et al. 1990 FEBS Lett. 270: 24-29; Weyland et al. 1994 Nature 368: 859-63). Although these genes were shown to be structurally related, different tissue-specific and species-specific expression of those genes was established (Distler et al. 1994 Neuropharmacology 33: 1275-82). For example, the full-length cDNA encoding the CNG channel polypeptide isolated from rabbit aorta was reported to be 93.7% homologous with bovine olfactory CNG polypeptide (Biel et al. 1993 FEBS Lett 329: 134-38). The functional role of the murine olfactory CNG polypeptide was established, in vivo, by constructing knockout mice lacking this gene. In these mutant mice, electrophysiological assays demonstrated that excitatory responses to odorants were undetectable, providing direct evidence for the role of this CNG channel in excitatory olfactory signal transduction (Brunet et al. 1996 Neuron 17: 682-93).

A second, distinct cDNA clone encoding a CNG channel α-subunit polypeptide was isolated initially from olfactory tissue (Dhallan et al. 1990 Nature 347: 184-87; Goulding et al. 1992 Neuron 8: 45-58; Ludwig et al. 1990 FEBS Lett. 270: 24-29) and later from rabbit aortic tissue (Biel et al. 1993 FEBS Lett. 329:134-38).

A third distinguishable cDNA clone encoding a CNG channel α-subunit polypeptide has also been cloned from both sensory and non-sensory tissues: cone photoreceptors (Bönigk et al. 1993 Neuron 10: 865-77), testis (Weyland et al. 1994 Nature 368: 859-63), and kidney tissue (Biel et al. 1994 Proc. Natl. Acad. Sci. 91: 3505-09).

Amino acid sequence comparisons between and among the encoded CNG α-subunit polypeptides identified above, as well observed regions of homology between these proteins and other ion channels polypeptides, have been used to construct a structural model for CNG α-subunit proteins (Zagotta et al. 1996 Ann. Rev. Neurosci. 19: 235-63). In this model, both the N-terminal and C-terminal sequences of CNG α-subunit polypeptide are positioned within the cell, and the termini of the α-subunit protein are separated by six transmembrane segments, designated S1 to S6 when viewed in the N-terminal to C-terminal direction. The peptide segment spanning the region between S5 and S6 constitutes the surface of the pore through which cations are conducted. 2 In addition, binding sites for Ca⁺²-Calmodulin and cAMP and/or cGMP have been identified on the intracellular N-terminal and C-terminal peptide segments, respectively. Heterologous expression of the above α-subunit polypeptide encoding CNG genes alone in, for example, Xenopus oocytes, provides a functional ion channel.

Clones have also been isolated that encode a second polypeptide subunit, referred to as the β-subunit polypeptide, of CNG channels (Chen et al. 1993; Bradley et al. 1994; Liman et al. 1994). Hydropathicity analyses of the two identified β-subunit polypeptides and amino acid sequence comparisons indicate that the β-subunit polypeptides, like the α-subunit polypeptides, consist of cytoplasmic amino- and carboxyl-termini separated by six transmembrane segments, a binding site for cyclic nucleotides within the C-terminal, intracellular portion of the protein, and an ion-conducting pore. Despite these structural similarities, there is only about a 40% amino acid sequence identity observed between the CNG α-subunit and β-subunit polypeptides, in contrast to the approximately 65% amino acid identity observed between the various CNG α-subunit polypeptide sequences. Furthermore, and in contrast to the results obtained with the α-subunit CNG polypeptide, heterologous expression of the β-subunit CNG polypeptide alone does not provide a functional ion channel. However, co-expression of both a and B CNG subunits yields heteromeric complexes having properties exhibited by naturally-occurring CNG channels that are not observed with homomeric CNG complexes formed with the α-subunit alone, including an increased affinity for cyclic-nucleotide binding. The β-subunit CNG polypeptides have, therefore, been referred to as modulatory subunits of CNG channels (Biel et al. 1999, Reviews of Physiology Biochemistry and Pharmacology 135: 151-71). Therefore, CNG channels consist of complexes of homologous but distinguishable α-subunits and β-subunits.

Kinetic models have been proposed which correlate cyclic nucleotide binding with CNG channel opening. In one model, summarized by Zagotta et al. (Zagotta et al. 1996 Ann. Rev. Neurosci. 19: 235-63), addition of cyclic nucleotides to four cooperative binding sites induces allosteric, conformational changes which result in the opening of the CNG channel. The existence of multiple, cooperative cyclic nucleotide binding sites forms the basis of the exquisite sensitivity of CNG channels to variations in the intracellular concentration of cAMP and/or cGMP.

Cyclic nucleotides serve as intracellular second messengers involved in regulated gene expression in response to extracellular signals. Such signals may be initiated, for example, by ligand binding to a G-protein coupled receptor, inducing conformational changes leading to intracellular activation of adenyl or guanyl cyclase. Resulting increases in the concentration of cyclic nucleotides can activate and open CNG channels, providing an influx of monovalent and/or divalent cations, and particularly calcium ions which, in turn, are directly involved in many aspects of biochemical and genetic regulation. It is through this biochemical cascade that CNG channels function as effector molecules for intracellular signals generated, for example, by G-protein coupled receptors.

Therefore, CNG channels are critical mediators of the cyclic nucleotide response generated in signal transduction pathways. The distribution of CNG channels within olfactory, auditory, brain, testicular, kidney, cardiac, and central nervous system tissues, demonstrates that CNG channels are important components of many critical biological processes. As such, human CNG channels are important targets, per se, for therapeutic intervention. Furthermore, CNG channels are also useful tools, in their role as effector molecules, for reflecting the modulation of intracellular cyclic nucleotide levels. Accordingly, CNG channels may also be used in assay procedures and screening methods for detection of compounds that modulate processes, including, but not limited to ligand binding and signal generation by G-protein coupled receptors, that affect intracellular cyclic nucleotide levels.

### 3. SUMMARY OF THE INVENTION

The present invention relates to the isolation and identification of nucleic acid molecules and proteins and polypeptides encoded by such nucleic acid molecules, or degenerate variants thereof, that participate in the formation or function of human ion channels. More specifically, the nucleic acid molecules of the invention include a novel human gene that encodes a protein or polypeptide involved in the formation or function of a novel cation channel.

According to one embodiment of the invention, a novel, complete human cDNA, termed HBMYCNG, and the amino acid sequence of its derived expressed protein, is disclosed.

The compositions of this invention include nucleic acid molecules, e.g., the HBMYCNG gene, including recombinant DNA molecules, cloned genes or degenerate variants thereof, especially naturally occurring variants, which encode the HBMYCNG gene product, and antibodies directed against that gene product or conserved variants or fragments thereof.

In particular, the compositions of the present invention include nucleic acid molecules (also referred to herein as “HBMYCNG nucleic acid molecules” or “HBMYCNG nucleic acids”) which comprise the following sequences: (a) nucleic acid sequences of the human HBMYCNG gene, e.g., as depicted in FIG. 1, and as deposited with the American Type Culture Collection (ATCC) as disclosed in Section 7, infra, as well as allelic variants and homologs thereof; (b) nucleic acid sequences that encode the HBMYCNG, gene product amino acid sequences, as depicted in FIG. 2; (c) nucleic acid sequences of a variant of the human HBMYCNG gene, e.g., as depicted in FIG. 5, and as deposited with the American Type Culture Collection (ATCC) as disclosed in Section 7, infra, as well as allelic variants and homologs thereof; (d) nucleic acid sequences that encode the variant HBMYCNG, gene product amino acid sequences, as depicted in FIG. 6; (e) nucleic acid sequences that encode portions of the HBMYCNG, gene product corresponding to functional domains and individual exons; (f) nucleic acid sequences comprising the novel complete gene sequence disclosed herein, or portions thereof, that encode mutants of the corresponding gene product in which all or a part of one or more of the domains is deleted or altered; (g) nucleic acid sequences that encode fusion proteins comprising the HBMYCNG gene product, or one or more of its domains, fused to a heterologous polypeptide; (h) nucleic acid sequences within the HBMYCNG gene, as well as chromosome sequences flanking that gene, that can be utilized as part of the methods of the present invention for the diagnosis or treatment of human disease; and (i) nucleic acid sequences that hybridize to the above-described sequences under stringent conditions. The nucleic acids of the invention include, but are not limited to, cDNA and genomic DNA sequences of the HBMYCNG gene.

The present invention also encompasses gene products of the nucleic acid molecules listed above; i.e., proteins and/or polypeptides that are encoded by the above-disclosed HBMYCNG nucleic acid molecules and are expressed in recombinant host systems.

Antagonists and agonists of the HBMYCNG gene and/or gene product disclosed herein are also included in the present invention. Such antagonists and agonists will include, for example, small molecules, large molecules, and antibodies directed against the HBMYCNG gene product. Antagonists and agonists of the invention also include nucleotide sequences, such as antisense and ribozyme molecules, and gene or regulatory sequence replacement constructs, that can be used to inhibit or enhance expression of the disclosed HBMYCNG nucleic acid molecules.

The present invention further encompasses cloning vectors, including expression vectors, that contain the nucleic acid molecules of the invention and can be used to express those nucleic acid molecules in host organisms. The present invention also relates to host cells engineered to contain and/or express the nucleic acid molecules of the invention. Further, host organisms that have been transformed with these nucleic acid molecules are also encompassed in the present invention, e.g., transgenic animals, particularly transgenic non-human animals, and more particularly transgenic non-human mammals.

The present invention also relates to methods and compositions for the diagnosis of human disease involving cation, e.g., Ca²⁺, sodium or potassium channel, dysfunction or lack of other ionic homeostasis including but not limited to, CNS disorders such as stroke, anxiety and depression, and degenerative neurological diseases, e.g., Alzheimer's disease or Parkinson's disease, or disorders such as cardiac disorders, e.g., arrhythmia, diabetes, chronic pain or other disorders such as hypercalcemia, hypercalciuria, or Ca²⁺, sodium or potassium channel dysfunction that is associated with immunological disorders (GI) tract disorders, or renal or liver disease. The present invention further relates to methods and compositions useful for the diagnosis and treatment of diseases and conditions related to or involving the serotonin nervous system which participates in the control of anxiety, fear, depression, sleep and pain. Accordingly, the present invention still further relates to methods and compositions for the diagnosis of anxiety and fear disorders, bipolar and major depression, panic disorder, headaches, migraine, disorders of circadian rhythmicity, stress, various sexual dysfunctions including but not limited to erectile dysfunction, neuroleptic-induced catalepsy, Rett syndrome and aggressive behaviors.

Such methods comprise, for example, measuring expression of the HBMYCNG gene in a patient sample, or detecting a mutation in the gene in the genome of a mammal, including a human, suspected of exhibiting ion channel dysfunction. The nucleic acid molecules of the invention can also be used as diagnostic hybridization probes or as primers for diagnostic PCR analysis to identify HBMYCNG gene mutations, allelic variations, or regulatory defects, such as defects in the expression of the gene. Such diagnostic PCR analyses can be used to diagnose individuals with disorders associated with a particular HBMYCNG gene mutation, allelic variation, or regulatory defect. Such diagnostic PCR analyses can also be used to identify individuals susceptible to ion channel disorders.

Methods and compositions, including pharmaceutical compositions, for the treatment of ion channel disorders are also included in the invention. Such methods and compositions are capable of modulating the level of HBMYCNG gene expression and/or the level of activity of the respective gene product. Such methods include, for example, modulating the expression of the HBMYCNG gene and/or the activity of the HBMYCNG gene product for the treatment of a disorder that is mediated by a defect in some other gene.

Such methods also include screening methods for the identification of compounds that modulate the expression of the nucleic acids and/or the activity of the polypeptides of the invention, e.g., assays that measure HBMYCNG mRNA and/or gene product levels, and assays that measure levels of HBMYCNG activity, such as the ability of the gene products to allow Ca²⁺ influx into cells.

For example, cellular and non-cellular assays are known that can be used to identify compounds that interact with the HBMYCNG gene and/or gene product, e.g., modulate the activity of the gene and/or bind to the gene product. Such cell-based assays of the invention utilize cells, cell lines, or engineered cells or cell lines that express the gene product.

In one embodiment, such methods comprise contacting a compound to a cell that expresses the HBMYCNG gene, measuring the level of gene expression, gene product expression, or gene product activity, and comparing this level to the level of the HBMYCNG gene expression, gene product expression, or gene product activity produced by the cell in the absence of the compound, such that if the level obtained in the presence of the compound differs from that obtained in its absence, a compound that modulates the expression of the HBMYCNG gene and/or the synthesis or activity of the gene product has been identified.

In an alternative embodiment, such methods comprise administering a compound to a host organism, e.g., a transgenic animal that expresses a HBMYCNG transgene or a mutant HBMYCNG transgene, and measuring the level of HBMYCNG gene expression, gene product expression, or gene product activity. The measured level is compared to the level of HBMYCNG gene expression, gene product expression, or gene product activity in a host that is not exposed to the compound, such that if the level obtained when the host is exposed to the compound differs from that obtained when the host is not exposed to the compound, a compound that modulates the expression of the HBMYCNG gene and/or the synthesis or activity of HBMYCNG gene products has been identified.

The compounds identified by these methods include therapeutic compounds that can be used as pharmaceutical compositions to reduce or eliminate the symptoms of ion channel disorders such as CNS disorders, e.g., stroke, chronic pain, anxiety and depression, or degenerative neurological diseases such as Alzheimer's disease or Parkinson's disease, cardiac diseases or other ion-related disorders such as hypercalcemia, hypocalcemia, hypercalciuria, hypocalciuria, or ion disorders that are associated with immunological disorders, gastrointestinal (GI) tract disorders, or renal or liver disease. Compounds identified by these methods further include compound useful for the treatment of diseases and conditions related to or involving the serotonin nervous system which participates in the control of anxiety, fear, depression, sleep and pain. Accordingly, compounds identified by these methods can be used for the treatment of anxiety and fear disorders, bipolar and major depression, panic disorder, headaches, migraine, disorders of circadian rhythmicity, stress, various sexual dysfunctions including but not limited to erectile dysfunction, neuroleptic-induced catalepsy, Rett syndrome and aggressive behaviors.

In another embodiment, screening methods are used for the detection, isolation, and identification of compounds which modulate the level of intracellular cyclic nucleotides. In one example, cells expressing the human HBMYCNG gene and a second biochemical activity involved in cyclic nucleotide synthesis or degradation, including but not limited to a G-protein coupled receptor, are contacted with a test compound and the level of calcium, or other cation, influx is determined. Evaluation of calcium, or other cation, influx in the presence or absence of the test compound indicates whether that compound is an agonist or antagonist of cyclic nucleotide accumulation within the cell.

Similarly, in another embodiment, such an assay can be used to detect, isolate, and characterize the cognate ligand recognized by an “orphan” G-protein coupled receptor. In this embodiment, the cell expressing both the human HBMYCNG gene and the orphan G-protein coupled receptor is contacted with compounds and/or mixtures of compounds, and human HBMYCNG mediated calcium, or other cation, influx is determined with and without the test compounds. Presence of the cognate ligand for the “orphan” receptor is indicated by the intracellular synthesis of cAMP and/or cGMP mediated by the activated G-protein coupled receptor, leading to activation of the HBMYCNG cation channel and an increase in calcium, or other cation, influx into the cell.

### 4. DESCRIPTION OF THE FIGURES

FIG. 1. Nucleotide sequence (SEQ ID NO:1) and amino acid sequence (SEQ ID NO:2) of the full length cDNA for Human HBMYCNG. The ATG initiation codon for HBMYCNG translation is found at nucleotides 20-22, and the TAA termination codon is found at nucleotides 2012-2014.

FIG. 2. Conceptual translation of the open reading frame of the cDNA sequence of FIG. 1, providing the amino acid sequence Human HBMYCNG (SEQ ID NO:2).

FIG. 3. Conceptual translation of nucleotide 20 to 2011 of the 2186-nucleotide (SEQ ID NO:2), full length Human HBMYCNG cDNA with the six transmembrane segments in bold and the ion pore underlined.

FIG. 4. Amino acid Sequence alignment of Human HBMYCNG (SEQ ID NO:2) and related rabbit (rACNG; gi 433960), bovine (CNG2_BOS; gi 227199), murine (CNG2_mouse; gi 6671780), and rat (CNG2_RAT; gi 227120) cyclic nucleotide gated channels. Blackened areas represent identical amino acids and the gray highlighted residues indicate similar amino acids.

FIG. 5. Nucleotide sequence (SEQ ID NO:23) and amino acid sequence (SEQ ID NO:24) of a variant of the full length cDNA for Human HBMYCNG. The ATG initiation codon for the variant HBMYCNG translation is found at nucleotides 20-22, and the TAA termination codon is found at nucleotides 2012-2014.

FIG. 6. Conceptual translation of the open reading frame of the cDNA sequence of FIG. 5, providing the amino acid sequence variant Human HBMYCNG (SEQ ID NO:24).

FIG. 7. Amino acid Sequence alignment of the Human HBMYCNG (SEQ ID NO:2) with the Human HBMYCNG variant (SEQ ID NO:24). Vertical bars (“I”) represent identical amino acids. The threonine to isoleucine amino acid change in the Human HBMYCNG variant sequence at amino acid position 442 of SEQ ID NO:24 is noted.

### 5. DETAILED DESCRIPTION OF THE INVENTION

The present invention relates to the isolation and identification of novel nucleic acid molecules and proteins and polypeptides for the formation or function of novel human ion channels. More specifically, the invention relates to a novel HBMYCNG human gene which encodes the corresponding HBMYCNG protein or biologically active derivatives or fragments thereof, involved in the formation or function of cation channels.

The HBMYCNG nucleic acid molecules of the present invention include isolated naturally-occurring or recombinantly-produced human HBMYCNG nucleic acid molecules, e.g., DNA molecules, cloned genes or degenerate variants thereof. The compositions of the invention also include isolated, naturally-occurring or recombinantly-produced human HBMYCNG protein or polypeptide.

Other embodiments of the invention include antibodies directed to the HBMYCNG protein or polypeptide of the invention and methods and compositions for the diagnosis and treatment of human diseases related to ion channel dysfunction as described below.

5.1. The HBMYCNG Nucleic Acids of the Invention

The complete HBMYCNG gene of the invention, HBMYCNG, is a novel, complete human nucleic acid molecule that encodes a protein or polypeptide involved in the formation or function of a novel human ion channel. Although this gene and the protein encoded therein display sequence and structural homology to other cation channel proteins known in the art, it is also known in the art that proteins displaying these homologies have significant differences in function, such as conductance and permeability, as well as differences in tissue expression, as well as co-expression, or not, of different CNG β-subunit polypeptides. As such, it is acknowledged in the art that nucleic acid molecules and the proteins encoded by those molecules sharing these homologies can still represent diverse, distinct and unique nucleic acids and proteins, respectively.

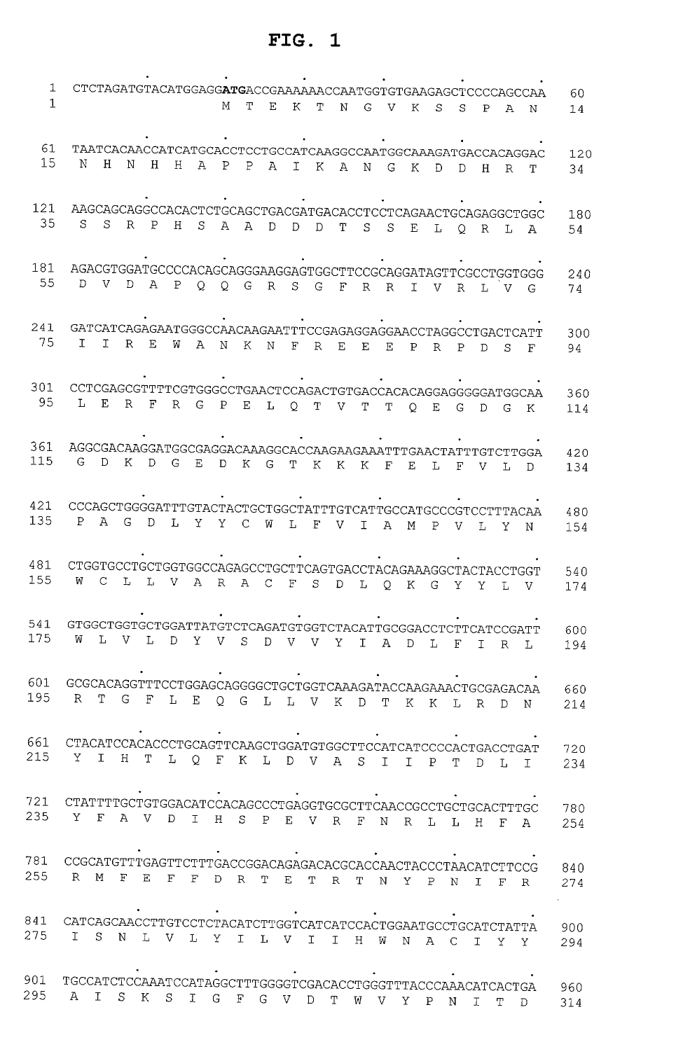

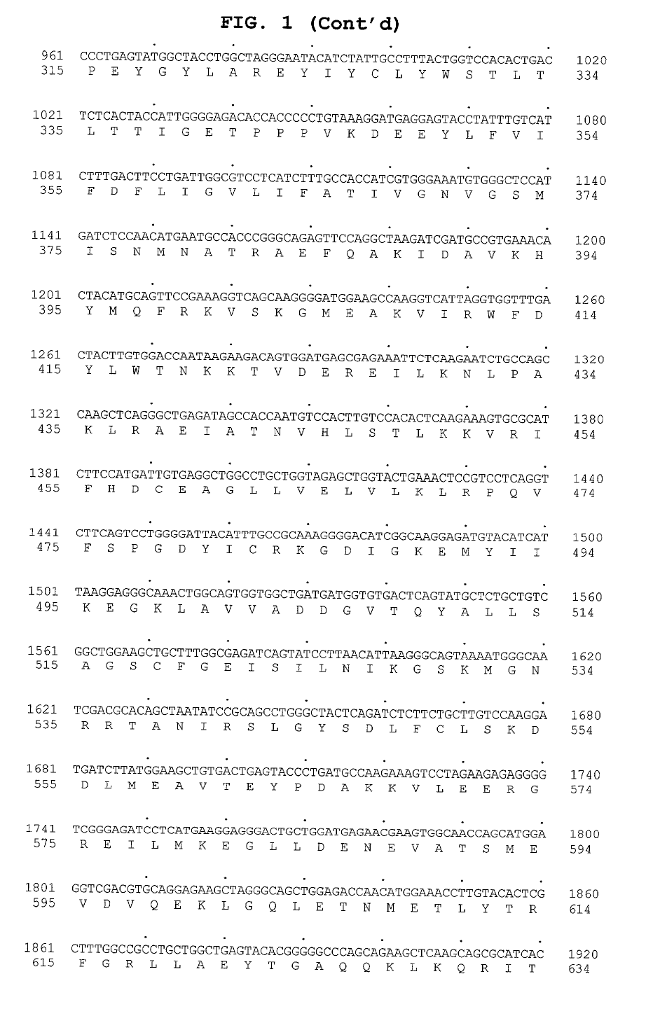

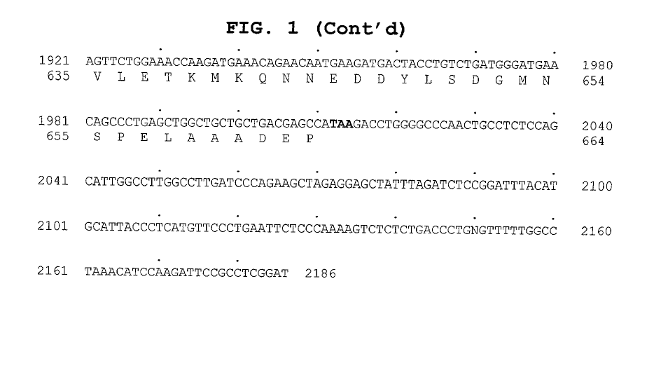

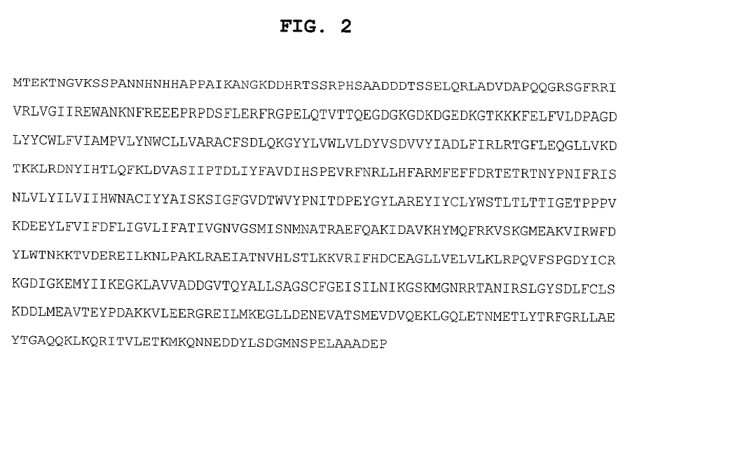

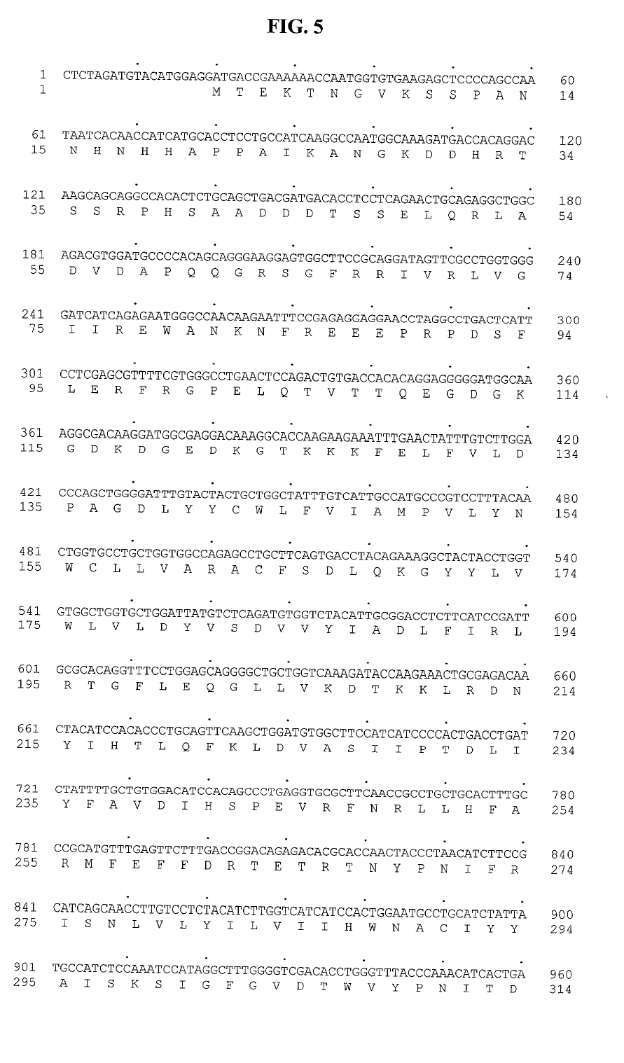

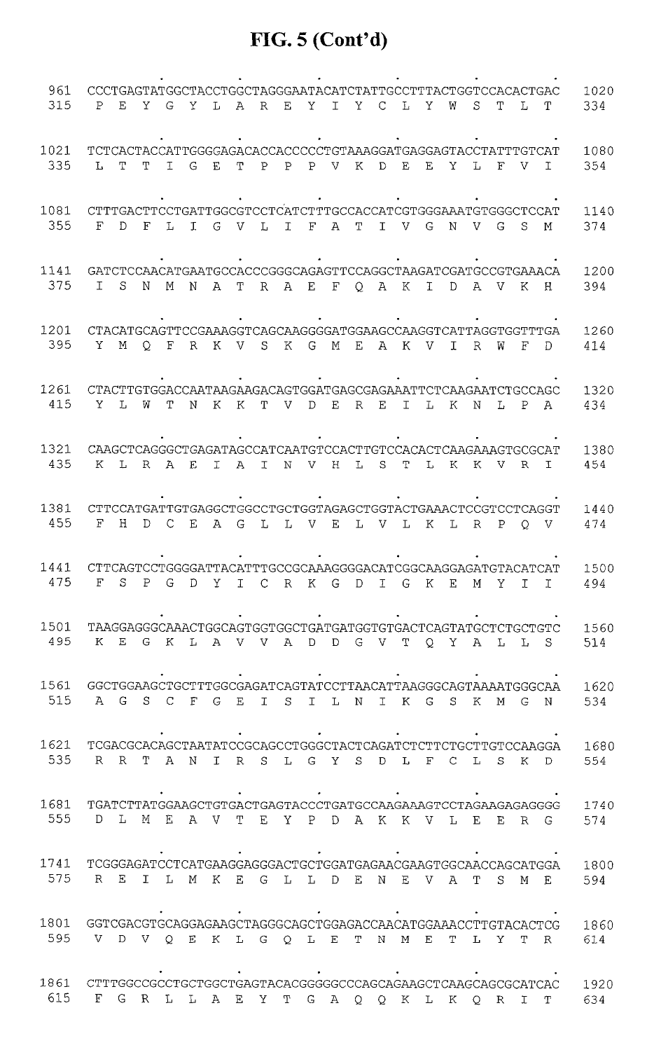

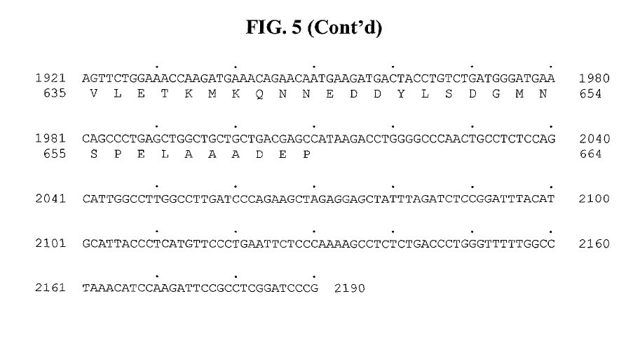

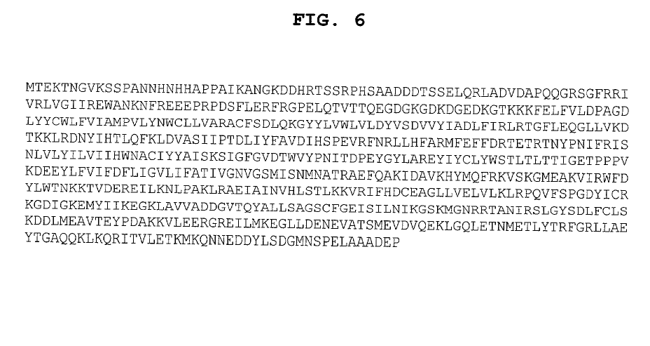

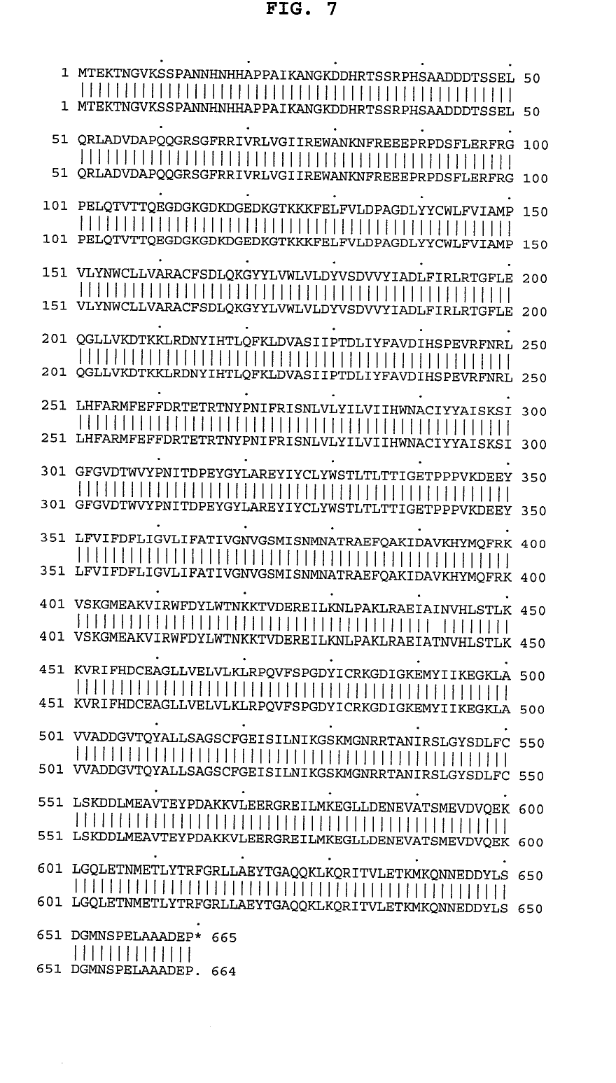

The HBMYCNG nucleic acid molecules of the invention include the following: (a) a nucleic acid molecule comprising the DNA sequence, HBMYCNG, as shown in FIG. 1 or FIG. 5; (b) any nucleic acid sequence that encodes the amino acid sequence, HBMYCNG as shown in FIG. 2 or FIG. 6; (c) any nucleic acid sequence that hybridizes to the complement of DNA sequences that encode the amino acid sequences of FIG. 2 or FIG. 6 under highly stringent conditions, e.g., hybridization to filter-bound DNA in 0.5 M NaHPO4, 7% sodium dodecyl sulfate (SDS), 1 mM EDTA at 65° C., and washing in 0.1× SSC/0.1% SDS at 68° C. (see, e.g., Ausubel F. M. et al., eds., 1989, Current Protocols in Molecular Biology, Vol. I, Green Publishing Associates, Inc., and John Wiley & sons, Inc., New York, at p. 2.10.3) or (d) any nucleic acid sequence that hybridizes to the complement of DNA sequences that encode the amino acid sequences of FIG. 2 or FIG. 6, under less stringent conditions, such as moderately stringent conditions, e.g., washing in 0.2× SSC/0.1% SDS at 42° C. (Ausubel et al., 1989, supra), and which encodes a gene product functionally equivalent to a HBMYCNG gene product encoded by the deposited sequences or the sequence depicted in FIG. 2 or FIG. 6. “Functionally equivalent” as used herein refers to any protein capable of exhibiting a substantially similar in vivo or in vitro activity as the HBMYCNG gene product encoded by the HBMYCNG nucleic acid molecules described herein, e.g., ion channel formation or function. For the purposes of the present invention, the HBMYCNG nucleic acid as depicted in FIG. 1 is functionally equivalent to the HBMYCNG nucleic acid as depicted in FIG. 5.

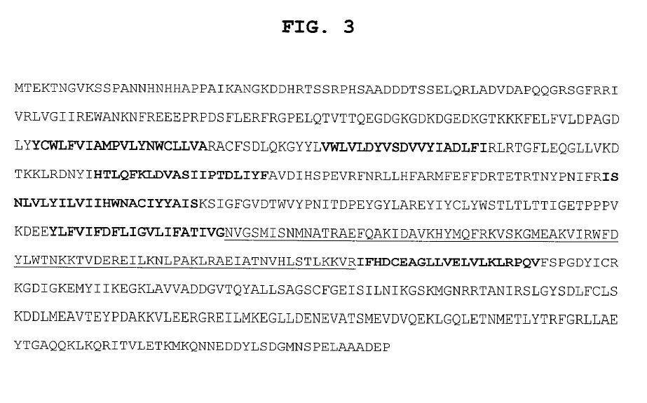

As used herein, the term “HBMYCNG nucleic acid molecule” may also refer to fragments and/or degenerate variants of DNA sequences (a) through (d), including naturally occurring variants or mutant alleles thereof. Such fragments include, for example, nucleotide sequences that encode portions of the HBMYCNG protein that correspond to functional domains of the protein. One embodiment of such a HBMYCNG nucleic acid fragment comprises a nucleic acid that encodes the fifth and sixth transmembrane segments of the HBMYCNG protein, including the predicted pore loop (see FIG. 3).

Additionally, the HBMYCNG nucleic acid molecules of the invention include isolated nucleic acid molecules, preferably DNA molecules, that hybridize under highly stringent or moderately stringent hybridization conditions to at least about 6, preferably at least about 12, and more preferably at least about 18, consecutive nucleotides of the nucleic acid sequences of (a) through (d), identified supra.

The HBMYCNG nucleic acid molecules of the invention also include nucleic acid molecules, preferably DNA molecules, that hybridize to, and are therefore complements of, the DNA sequences of (a) through (d), supra. Such hybridization conditions may be highly stringent or moderately stringent, as described above. In those instances in which the nucleic acid molecules are deoxyoligonucleotides (“oligos”), highly stringent conditions may include, e.g., washing in 6× SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as HBMYCNG antisense molecules useful, for example, in HBMYCNG gene regulation or as antisense primers in amplification reactions of HBMYCNG nucleic acid sequences. Further, such sequences may be used as part of ribozyme and/or triple helix sequences, also useful for HBMYCNG gene regulation. Still further, such molecules may be used as components of diagnostic methods whereby, for example, the presence of a particular HBMYCNG allele or alternatively spliced HBMYCNG transcript responsible for causing or predisposing one to a disorder involving ion channel dysfunction may be detected.

Typically, the HBMYCNG nucleic acids of the invention should exhibit at least about 90% overall homology at the nucleotide level, and more preferably at least about 95% overall homology to the nucleic acid sequence of FIG. 1.

Also included within the HBMYCNG nucleic acids of the invention are nucleic acid molecules, preferably DNA molecules, comprising an HBMYCNG nucleic acid, as described herein, operatively linked to a nucleotide sequence encoding a heterologous protein or peptide.

To determine the percent identity of two nucleic acid sequences or of two amino acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleic acid searches can be performed with the NBLAST nucleic acid program parameters set, e.g., for score=100, wordlength=12 to obtain nucleic acid sequences homologous to a nucleic acid molecule of the present invention. BLAST polypeptide searches can be performed with the XBLAST program parameters set, e.g., to score-SO, wordlength=3 to obtain amino acid sequences homologous to a polypeptide molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Still another preferred algorithm for the comparison of polypeptide sequences is that of Thompson et al., designated CLUSTALW, which is disclosed in Thompson et al. 1994 Nucleic Acids Research 2(22): 4673-80.

The percent identity between two sequences can be determined using techniques similar to those described above, with or without allowing gaps. In calculating percent identity, typically only exact matches are counted.

Moreover, due to the degeneracy of the genetic code, other DNA sequences that encode substantially the amino acid sequence of HBMYCNG may be used in the practice of the present invention for the cloning and expression of HBMYCNG polypeptides. Such DNA sequences include those that are capable of hybridizing to the HBMYCNG nucleic acids of this invention under stringent (high or moderate) conditions, or that would be capable of hybridizing under stringent conditions but for the degeneracy of the genetic code.

Altered HBMYCNG DNA sequences that may be used in accordance with the invention include deletions, additions or substitutions of different nucleotide residues resulting in a nucleic acid molecule that encodes the same or a functionally equivalent gene product as those described supra. The gene product itself may contain deletions, additions or substitutions of amino acid residues within the HBMYCNG protein sequence, which result in a silent change, thus producing a functionally equivalent HBMYCNG polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine and arginine; amino acids with uncharged polar head groups having similar hydrophilicity values include the following: leucine, isoleucine, valine; glycine, aniline; asparagine, glutamine; serine, threonine; phenylalanine, tyrosine. A functionally equivalent HBMYCNG polypeptide can include a polypeptide which displays the same type of biological activity (e.g., cation channel) as the native HBMYCNG protein, but not necessarily to the same extent.

The nucleic acid molecules or sequences of the invention may be engineered in order to alter the HBMYCNG coding sequence for a variety of ends including but not limited to alterations that modify processing and expression of the gene product. For example, mutations may be introduced using techniques which are well known in the art, e.g., site-directed mutagenesis, to insert new restriction sites, to alter glycosylation patterns, phosphorylation, etc. For example, in certain expression systems such as yeast, host cells may over-glycosylate the gene product. When using such expression systems, it may be preferable to alter the HBMYCNG coding sequence to eliminate any N-linked glycosylation sites.

In another embodiment of the invention, the HBMYCNG nucleic acid or a modified HBMYCNG sequence may be ligated to a heterologous sequence to encode a fusion protein. The fusion protein may be engineered to contain a cleavage site located between the HBMYCNG sequence and the heterologous protein sequence, so that the HBMYCNG protein can be cleaved away from the heterologous moiety.

The HBMYCNG nucleic acid molecules of the invention can also be used as hybridization probes for obtaining HBMYCNG cDNAs or genomic HBMYCNG DNA. In addition, the nucleic acids of the invention can be used as primers in PCR amplification methods to isolate HBMYCNG cDNAs and genomic DNA, e.g., from other species.

The HBMYCNG gene sequences of the invention may also used to isolate mutant HBMYCNG gene alleles. Such mutant alleles may be isolated from individuals either known or proposed to have a genotype related to ion channel dysfunction. Mutant alleles and mutant allele gene products may then be utilized in the screening, therapeutic and diagnostic systems described in Section 5.4., infra. Additionally, such HBMYCNG gene sequences can be used to detect HBMYCNG gene regulatory (e.g., promoter) defects which can affect ion channel function.

A cDNA of a mutant HBMYCNG gene may be isolated, for example, by using PCR, a technique which is well known to those of skill in the art (see, e.g., U.S. Pat. No. 4,683,202). The first cDNA strand may be synthesized by hybridizing an oligo-dT oligonucleotide to mRNA isolated from tissue known or suspected to be expressed in an individual putatively carrying the mutant HBMYCNG allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known in the art. By comparing the DNA sequence of the mutant HBMYCNG allele to that of the normal HBMYCNG allele, the mutation(s) responsible for the loss or alteration of function of the mutant HBMYCNG gene product can be ascertained.

Alternatively, a genomic library can be constructed using DNA obtained from an individual suspected of or known to carry the mutant HBMYCNG allele, or a cDNA library can be constructed using RNA from a tissue known, or suspected, to express the mutant HBMYCNG allele. The normal HBMYCNG gene or any suitable fragment thereof may then be labeled and used as a probe to identify the corresponding mutant HBMYCNG allele in such libraries. Clones containing the mutant HBMYCNG gene sequences may then be purified and subjected to sequence analysis according to methods well known in the art.

According to another embodiment, an expression library can be constructed utilizing cDNA synthesized from, for example, RNA isolated from a tissue known, or suspected, to express a mutant HBMYCNG allele in an individual suspected of or known to carry such a mutant allele. Gene products made by the putatively mutant tissue may be expressed and screened using standard antibody screening techniques in conjunction with antibodies raised against the normal HBMYCNG gene product, as described in Section 5.3, supra. For screening techniques, see, for example, Harlow, E. and Lane, eds., 1988, “Anti-bodies: A Laboratory Manual”, Cold Spring Harbor Press, Cold Spring Harbor.

In cases where a HBMYCNG mutation results in an expressed gene product with altered function (e.g., as a result of a missense or a frameshift mutation), a polyclonal set of anti-HBMYCNG gene product antibodies are likely to cross-react with the mutant HBMYCNG gene product. Library clones detected via their reaction with such labeled antibodies can be purified and subjected to sequence analysis according to methods well known to those of skill in the art.

In an alternate embodiment of the invention, the coding sequence of HBMYCNG can be synthesized in whole or in part, using chemical methods well known in the art, based on the nucleic acid and/or amino acid sequences of the HBMYCNG genes and proteins disclosed herein. See, for example, Caruthers et al., 1980, Nuc. Acids Res. Symp. Ser. 7: 215-233; Crea and Horn, 1980, Nuc. Acids Res. 9(10): 2331; Matteucci and Caruthers, 1980, Tetrahedron Letters 21: 719; and Chow and Kempe, 1981, Nuc. Acids Res. 9(12): 2807-2817.

The invention also encompasses (a) DNA vectors that contain any of the foregoing HBMYCNG sequences and/or their complements; (b) DNA expression vectors that contain any of the foregoing HBMYCNG coding sequences operatively associated with a regulatory element that directs the expression of the coding sequences; and (c) genetically engineered host cells that contain any of the foregoing HBMYCNG coding sequences operatively associated with a regulatory element that directs the expression of the coding sequences in the host cell. As used herein, regulatory elements include, but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention still further includes nucleic acid analogs, including but not limited to peptide nucleic acid analogues, equivalent to the nucleic acid molecules described herein. “Equivalent” as used in this context refers to nucleic acid analogs that have the same primary base sequence as the nucleic acid molecules described above. Nucleic acid analogs and methods for the synthesis of nucleic acid analogs are well known to those of skill in the art. See, e.g., Egholm, M. et al., 1993, Nature 365:566-568; and Perry-O'Keefe, H. et al., 1996, Proc. Natl. Acad. USA 93:14670-14675.

5.2. HBMYCNG Proteins and Polypeptides of the Invention

The HBMYCNG nucleic acid molecules of the invention may be used to generate recombinant DNA molecules that direct the expression in appropriate host cells of HBMYCNG polypeptides, including the full-length HBMYCNG protein, functionally active or equivalent HBMYCNG proteins and polypeptides, e.g., mutated, truncated or deleted forms of HBMYCNG, peptide fragments of HBMYCNG, or HBMYCNG fusion proteins. A functionally equivalent HBMYCNG polypeptide can include a polypeptide which displays the same type of biological activity (e.g., cation channel formation and/or function) as the native HBMYCNG protein, but not necessarily to the same extent.

In a preferred embodiment, the proteins and polypeptides of the invention include the HBMYCNG amino acid sequence depicted in FIG. 2, which corresponds to the conceptual translation of the nucleotide sequence spanning residues 20 to 2011 of the cDNA sequence of HBMYCNG, as depicted in FIG. 1. This amino acid sequence includes six transmembrane domains and an overall topology that is conserved in CNG ion channels.

In other embodiments of the present invention the proteins and polypeptides of the invention include the HBMYCNG amino acid sequence depicted in FIG. 2 except for the initial methionine residue; i.e., a polypeptide having an amino acid sequence corresponding to amino acids 2 through 664 the amino acid sequence of FIG. 2, which corresponds to the conceptual translation of the nucleotide sequence spanning residues 23 to 2011 of the cDNA sequence of HBMYCNG, as depicted in FIG. 1.

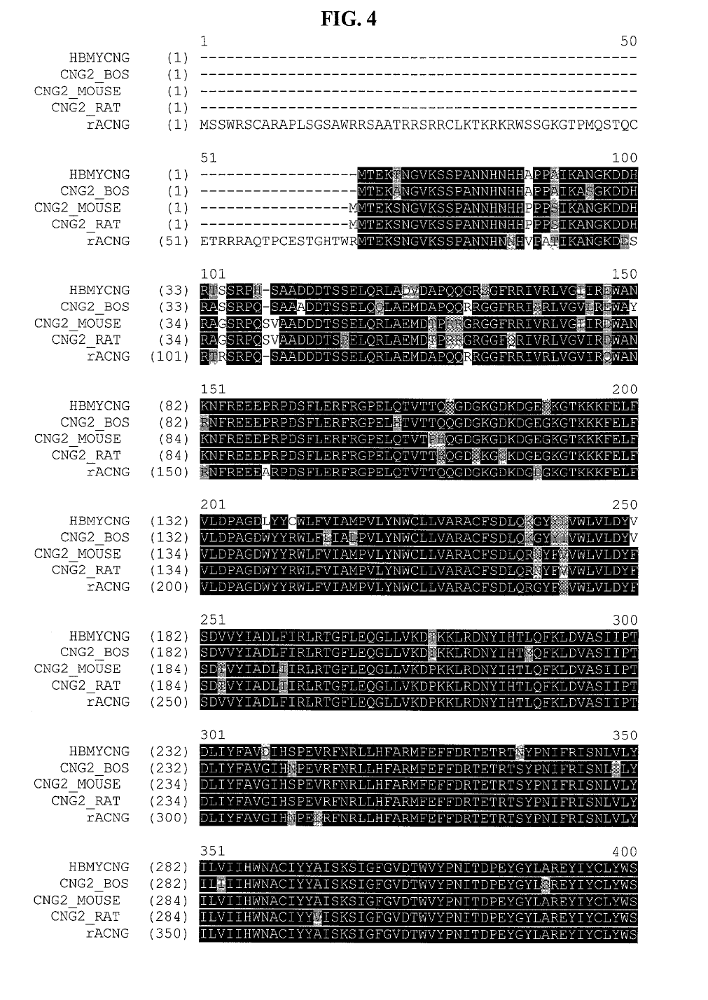

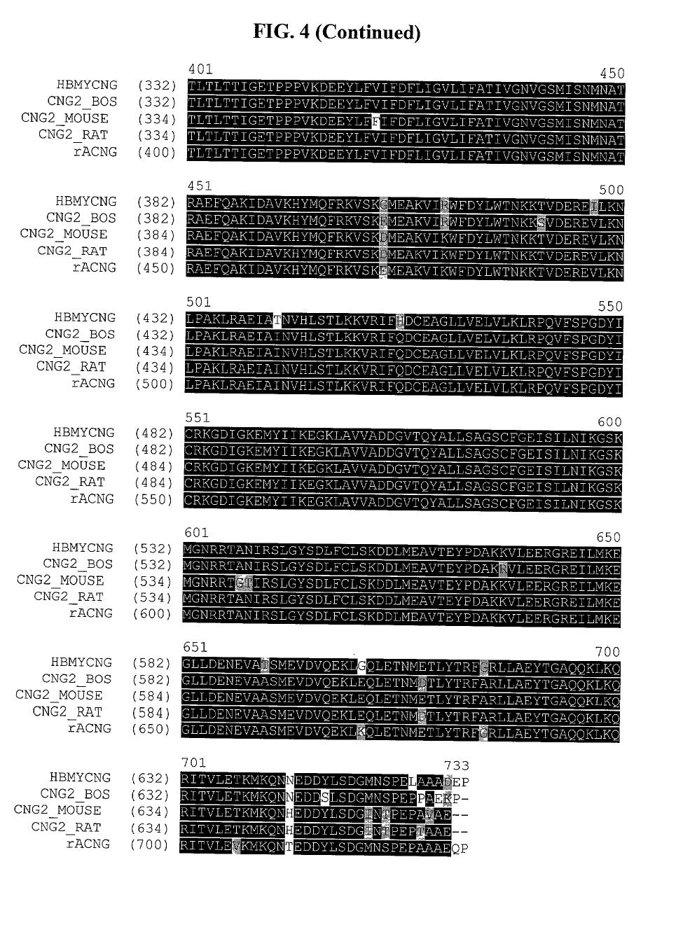

The HBMYCNG amino acid sequence of FIG. 2, which has a calculated molecular weight of 75.9 kDa, is homologous to four cyclic nucleotide gated proteins. A comparison of the HBMYCNG amino acid sequence with that of rabbit (rACNG; gi 433960), bovine (CNG2_BOS; gi 227199), mouse (CNG2_mouse; gi 6671780), and rat (CNG2_RAT; gi 227120) cyclic nucleotide gated channels from rabbit is presented in FIG. 4. The amino acid sequences for Human HBMYCNG and for rabbit aorta rCNG displayed 95.633% similarity and 93.675% identity; the amino acid sequences for Human HBMYCNG and for bovine olfactory CNG2_BOVIN displayed 95.324% similarity and 93.213% identity; the amino acid sequences for Human HBMYCNG and for murine olfactory CNG2_MOUSE displayed 94.260% similarity and 93.051% identity; and the amino acid sequences for Human HBMYCNG and for rat olfactory CNG2 RAT displayed 94.109% similarity and 92.598% identity.

The HBMYCNG proteins and polypeptides of the invention include peptide fragments of HBMYCNG, e.g., peptides corresponding to one or more domains of the protein, mutated, truncated or deleted forms of the proteins and polypeptides, as well as HBMYCNG fusion proteins, all of which derivatives of HBMYCNG can be obtained by techniques well known in the art, given the HBMYCNG nucleic acid and amino acid sequences disclosed herein.

As noted in Section 5.1, supra, the proteins and polypeptides of the invention may contain deletions, additions or substitutions of amino acid residues within the HBMYCNG protein sequence, which result in a silent change, thus producing a functionally equivalent HBMYCNG polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine, arginine and histidine; amino acids with uncharged polar head groups having similar hydrophilicity values include the following: leucine, isoleucine, valine, glycine, alanine, asparagine, glutamine, serine, threonine, phenylalanine, tyrosine.

Mutated or altered forms of the HBMYCNG proteins and polypeptides of the invention can be obtained using either random mutagenesis techniques or site-directed mutagenesis techniques well known in the art or by chemical methods, e.g., protein synthesis techniques (see Section 5.1, supra). Mutant HBMYCNG proteins or polypeptides can be engineered so that regions important for function are maintained, while variable residues are altered, e.g., by deletion or insertion of an amino acid residue(s) or by the substitution of one or more different amino acid residues. For example, conservative alterations at the variable positions of a polypeptide can be engineered to produce a mutant HBMYCNG polypeptide that retains the function of HBMYCNG. Non-conservative alterations of variable regions can be engineered to alter HBMYCNG function, if desired. Alternatively, in those cases where modification of function (either to increase or decrease function) is desired, deletion or non-conservative alterations of conserved regions of the polypeptide may be engineered.

Fusion proteins containing HBMYCNG amino acid sequences can also be obtained by techniques known in the art, including genetic engineering and chemical protein synthesis techniques. According to a preferred embodiment, the fusion proteins of the invention are encoded by an isolated nucleic acid molecule comprising an HBMYCNG nucleic acid of the invention that encodes a polypeptide with an activity of a HBMYCNG protein, or a fragment thereof, linked in frame and uninterrupted by stop codons to a nucleotide sequence that encodes a heterologous protein or peptide.

The fusion proteins of the invention include those that contain the full length HBMYCNG amino acid sequence, an HBMYCNG peptide sequence, e.g., encoding one or more functional domains, a mutant HBMYCNG amino acid sequence or a truncated HBMYCNG amino acid sequence linked to an unrelated protein or polypeptide sequence. Such fusion proteins include but are not limited to IgFc fusions which stabilize the HBMYCNG fusion protein and may prolong half-life of the protein in vivo or fusions to an enzyme, fluorescent protein or luminescent protein that provides a marker function.

According to a preferred embodiment, the HBMYCNG nucleic acid molecules of the invention may be used to generate recombinant DNA molecules that direct the expression of HBMYCNG polypeptides, including the full-length HBMYCNG protein, e.g., HBMYCNG or functionally active or equivalent HBMYCNG peptides thereof, or HBMYCNG fusion proteins in appropriate host cells.

In order to express a biologically active HBMYCNG polypeptide, a nucleic acid molecule coding for the polypeptide, or a functional equivalent thereof as described in Section 5.1, supra, is inserted into an appropriate expression vector, i.e., a vector which contains the necessary elements for the transcription and translation of the inserted coding sequence. The HBMYCNG gene products so produced, as well as host cells or cell lines transfected or transformed with recombinant HBMYCNG expression vectors, can be used for a variety of purposes. These include but are not limited to generating antibodies (i.e., monoclonal or polyclonal) that bind to the HBMYCNG protein, including those that competitively inhibit binding and thus can “neutralize” HBMYCNG activity, and the screening and selection of HBMYCNG analogs or ligands.

Methods which are well known to those skilled in the art are used to construct expression vectors containing the HBMYCNG coding sequences of the invention and appropriate transcriptional and translational control signals. These methods include in vitro recombinant DNA techniques, synthetic techniques and in vivo recombination/genetic recombination. See, for example, the techniques described in Maniatis et al., 1989, Molecular Cloning, A Laboratory Manual, Cold Spring Harbor Laboratory, N.Y. and Ausubel et al., 1989, Current Protocols in Molecular Biology, Greene Publishing Associates and Wiley Interscience, N.Y. See also Sambrook et al., 1989, Molecular Cloning, A Laboratory Manual, Cold Spring Harbor Press, N.Y.

A variety of host-expression vector systems may be used to express the HBMYCNG coding sequences of this invention. Such host-expression systems represent vehicles by which the coding sequences of interest may be produced and subsequently purified, but also represent cells which may, when transformed or transfected with the appropriate nucleotide coding sequences, exhibit the corresponding HBMYCNG gene products in situ and/or function in vivo. These hosts include but are not limited to microorganisms such as bacteria (e.g., E. coli, B. subtilis) transformed with recombinant bacteriophage DNA, plasmid DNA or cosmid DNA expression vectors containing the HBMYCNG coding sequences; yeast (e.g., Saccharomyces, Pichia) transformed with recombinant yeast expression vectors containing the HBMYCNG coding sequence; insect cell systems infected with recombinant virus expression vectors (e.g., baculovirus) containing the HBMYCNG coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the HBMYCNG coding sequence; or mammalian cell systems (e.g., COS, CHO, BHK, 293, 3T3) harboring recombinant expression constructs containing promoters derived from the genome of mammalian cells (e.g., the metallothionein promoter) or from mammalian viruses (e.g., the adenovirus late promoter or vaccinia virus 7.5K promoter).

The expression elements of these systems can vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage ?, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the HBMYCNG DNA, SV40-, BPV- and EBV-based vectors may be used with an appropriate selectable marker.

In bacterial systems, a number of expression vectors may be advantageously selected depending upon the use intended for the HBMYCNG expressed. For example, when large quantities of an HBMYCNG polypeptide are to be produced, e.g., for the generation of antibodies or the production of the HBMYCNG gene product, vectors which direct the expression of high levels of fusion protein products that are readily purified may be desirable. Such vectors include but are not limited to the E. coli expression vector pUR278 (Ruther et al., 1983, EMBO J. 2: 1791), in which the HBMYCNG coding sequence may be ligated into the vector in frame with the lacZ coding region so that a hybrid HBMYCNG/lacZ protein is produced; pIN vectors (Inouye & Inouye, 1985, Nucleic Acids Res. 13: 3101-3109; Van Heeke & Schuster, 1989, J. Biol. Chem. 264: 5503-5509); and the like. pGEX vectors may also be used to express foreign polypeptides as fusion proteins with glutathione S-transferase (GST). In general, such fusion proteins are soluble and can easily be purified from lysed cells by affinity chromatography, e.g., adsorption to glutathione-agarose beads followed by elution in the presence of free glutathione. The pGEX vectors are designed to include thrombin or factor Xa protease cleavage sites so that the cloned polypeptide of interest can be released from the GST moiety. See also Booth et al., 1988, Immunol. Lett. 19: 65-70; and Gardella et al., 1990, J. Biol. Chem. 265: 15854-15859; Pritchett et al., 1989, Biotechniques 7: 580.

In yeast, a number of vectors containing constitutive or inducible promoters may be used. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Cold Spring Harbor Press, Vols. I and II.

In an insect system, Autographa californica nuclear polyhidrosis virus (AcNPV) can be used as a vector to express foreign genes. The virus grows in Spodoptera frugiperda cells. The HBMYCNG coding sequence may be cloned into non-essential regions (for example, the polyhedrin gene) of the virus and placed under control of an AcNPV promoter (for example, the polyhedrin promoter). Successful insertion of the HBMYCNG coding sequence will result in inactivation of the polyhedrin gene and production of non-occluded recombinant virus (i.e., virus lacking the proteinaceous coat coded for by the polyhedrin gene). These recombinant viruses can then be used to infect Spodoptera frugiperda cells in which the inserted gene is expressed (see e.g., Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the HBMYCNG coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing HBMYCNG in infected hosts (see, e.g., Logan & Shenk, 1984, Proc. Natl. Acad. Sci. (USA) 81: 3655-3659). Alternatively, the vaccinia 7.5K promoter may be used (see, e.g., Mackett et al., 1982, Proc. Natl. Acad. Sci. (USA) 79: 7415-7419; Mackett et al., 1984, J. Virol. 49: 857-864; Panicali et al., 1982, Proc. Natl. Acad. Sci. 79: 4927-4931).

Specific initiation signals may also be required for efficient translation of inserted HBMYCNG coding sequences. These signals include the ATG initiation codon and adjacent sequences. In cases where the entire HBMYCNG gene, including its own initiation codon and adjacent sequences, is inserted into the appropriate expression vector, no additional translational control signals may be needed. However, in cases where only a portion of the HBMYCNG coding sequence is inserted, exogenous translational control signals, including the ATG initiation codon, must be provided. Furthermore, the initiation codon must be in phase with the reading frame of the HBMYCNG coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, WI38, etc.

For long-term, high-yield production of recombinant proteins, stable expression is preferred. For example, cell lines which stably express the HBMYCNG polypeptides of this invention may be engineered. Thus, rather than using expression vectors which contain viral origins of replication, host cells can be transformed with HBMYCNG nucleic acid molecules, e.g., DNA, controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express HBMYCNG polypeptides on the cell surface. Such engineered cell lines are particularly useful in screening for HBMYCNG analogs or ligands.

In instances where the mammalian cell is a human cell, among the expression systems by which the HBMYCNG nucleic acid sequences of the invention can be expressed are human artificial chromosome (HAC) systems (see, e.g., Harrington et al., 1997, Nature Genetics 15: 345-355).

HBMYCNG gene products can also be expressed in transgenic animals such as mice, rats, rabbits, guinea pigs, pigs, micro-pigs, sheep, goats, and non-human primates, e.g., baboons, monkeys, and chimpanzees. The term “transgenic” as used herein refers to animals expressing HBMYCNG nucleic acid sequences from a different species (e.g., mice expressing human HBMYCNG nucleic acid sequences), as well as animals that have been genetically engineered to overexpress endogenous (i.e., same species) HBMYCNG nucleic acid sequences or animals that have been genetically engineered to no longer express endogenous HBMYCNG nucleic acid sequences (i.e., “knock-out” animals), and their progeny.

Transgenic animals according to this invention may be produced using techniques well known in the art, including but not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82: 6148-6152); gene targeting in embryonic stem cells (Thompson et al., 1989, Cell 56: 313-321); electroporation of embryos (Lo, 1983, Mol Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229.

In addition, any technique known in the art may be used to produce transgenic animal clones containing a HBMYCNG transgene, for example, nuclear transfer into enucleated oocytes of nuclei from cultured embryonic, fetal or adult cells induced to quiescence (Campbell et al., 1996, Nature 380: 64-66; Wilmut et al., 1997, Nature 385: 810-813).

Host cells which contain the HBMYCNG coding sequence and which express a biologically active gene product may be identified by at least four general approaches; (a) DNA-DNA or DNA-RNA hybridization; (b) the presence or absence of “marker” gene functions; (c) assessing the level of transcription as measured by the expression of HBMYCNG mRNA transcripts in the host cell; and (d) detection of the gene product as measured by immunoassay or by its biological activity.

In the first approach, the presence of the HBMYCNG coding sequence inserted in the expression vector can be detected by DNA-DNA or DNA-RNA hybridization using probes comprising nucleotide sequences that are homologous to the HBMYCNG coding sequence, respectively, or portions or derivatives thereof.

In the second approach, the recombinant expression vector/host system can be identified and selected based upon the presence or absence of certain “marker” gene functions. For example, if the HBMYCNG coding sequence is inserted within a marker gene sequence of the vector, recombinants containing the HBMYCNG coding sequence can be identified by the absence of the marker gene function. Alternatively, a marker gene can be placed in tandem with the HBMYCNG sequence under the control of the same or different promoter used to control the expression of the HBMYCNG coding sequence. Expression of the marker in response to induction or selection indicates expression of the HBMYCNG coding sequence.

Selectable markers include resistance to antibiotics, resistance to methotrexate, transformation phenotype, and occlusion body formation in baculovirus. In addition, thymidine kinase activity (Wigler et al., 1977, Cell 11: 223) hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk-, hgprt- or aprt-cells, respectively. Also, antimetabolite resistance can be used as the basis of selection for dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 (Colberre-Garapin, et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85: 8047); and ODC (ornithine decarboxylase) which confers resistance to the ornithine decarboxylase inhibitor, 2-(difluoromethyl)-DL-ornithine, DFMO (McConlogue, 1987, in Current Communications in Molecular Biology, Cold Spring Harbor Laboratory ed.).

In the third approach, transcriptional activity for the HBMYCNG coding region can be assessed by hybridization assays. For example, RNA can be isolated and analyzed by Northern blot using a probe homologous to the HBMYCNG coding sequence or particular portions thereof. Alternatively, total nucleic acids of the host cell may be extracted and assayed for hybridization to such probes.

In the fourth approach, the expression of the HBMYCNG protein product can be assessed immunologically, for example by Western blots, immunoassays such as radioimmuno-precipitation, enzyme-linked immunoassays and the like. The ultimate test of the success of the expression system, however, involves the detection of biologically active HBMYCNG gene product. A number of assays can be used to detect HBMYCNG activity including but not limited to binding assays and biological assays for HBMYCNG activity.

Once a clone that produces high levels of a biologically active HBMYCNG polypeptide is identified, the clone may be expanded and used to produce large amounts of the polypeptide which may be purified using techniques well known in the art, including but not limited to, immunoaffinity purification using antibodies, immunoprecipitation or chromatographic methods including high performance liquid chromatography (HPLC).

Where the HBMYCNG coding sequence is engineered to encode a cleavable fusion protein, purification may be readily accomplished using affinity purification techniques. For example, a collagenase cleavage recognition consensus sequence may be engineered between the carboxy terminus of HBMYCNG and protein A. The resulting fusion protein may be readily purified using an IgG column that binds the protein A moiety. Unfused HBMYCNG may be readily released from the column by treatment with collagenase. Another example would be the use of pGEX vectors that express foreign polypeptides as fusion proteins with glutathionine S-transferase (GST). The fusion protein may be engineered with either thrombin or factor Xa cleavage sites between the cloned gene and the GST moiety. The fusion protein may be easily purified from cell extracts by adsorption to glutathione agarose beads followed by elution in the presence of glutathione. In fact, any cleavage site or enzyme cleavage substrate may be engineered between the HBMYCNG gene product sequence and a second peptide or protein that has a binding partner which could be used for purification, e.g., any antigen for which an immunoaffinity column can be prepared.

In addition, HBMYCNG fusion proteins may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

In another embodiment, fusion proteins comprising at least one extracellular domain (i.e. the extracellular domains consist approximately of amino acid residues 161-173, 237-274, and 370-453) of the HMBYCNG polypeptide are expressed from a genetically-engineered gene constructed and expressed using any recombinant method described above. In one aspect of this embodiment, a “soluble” derivative of the HMBYCNG protein is synthesized within which the six transmembrane domains (represented by amino acid residues 141-160, 174-192, 217-236, 275-297, 350-369, and 454-474 of the protein sequence of FIG. 3) are replaced with peptide sequences of comparable length and structure, providing a water soluble fusion protein mimic of the HMBYCNG polypeptide.

Alternatively, the HBMYCNG protein itself can be produced using chemical methods to synthesize the HBMYCNG amino acid sequence in whole or in part. For example, peptides can be synthesized by solid phase techniques, cleaved from the resin, and purified by preparative high performance liquid chromatography (see, e.g., Creighton, 1983, Proteins Structures And Molecular Principles, W. H. Freeman and Co., N.Y., pp. 50-60). The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

The HBMYCNG proteins, polypeptides and peptide fragments, mutated, truncated or deleted forms of HBMYCNG and/or HBMYCNG fusion proteins can be prepared for various uses, including but not limited to, the generation of antibodies, as reagents in diagnostic assays, the identification of other cellular gene products involved in ion transport, as reagents in assays for screening for compounds for use in the treatment of ion channel disorders.

5.3. Antibodies to HBMYCNG Polypeptides

The present invention also includes antibodies directed to the HBMYCNG polypeptides of this invention and methods for the production of those antibodies, including antibodies that specifically recognize one or more HBMYCNG epitopes or epitopes of conserved variants or peptide fragments of HBMYCNG, or antibodies which recognize the extracellular domains of the CNG α-subunit polypeptides, or which recognize HBMYCNG epitopes within the water soluble fusion protein mimic of the HMBYCNG polypeptide disclosed above.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a HBMYCNG protein or polypeptide in an biological sample and may, therefore, be utilized as part of a diagnostic or prognostic technique whereby patients may be tested for abnormal levels of HBMYCNG and/or for the presence of abnormal forms of the protein. Such antibodies may also be utilized in conjunction with, for example, compound screening protocols for the evaluation of the effect of test compounds on HBMYCNG levels and/or activity. Additionally, such antibodies can be used in conjunction with the gene therapy techniques described in Section 5.4, infra, to, for example, evaluate normal and/or genetically-engineered HBMYCNG-expressing cells prior to their introduction into the patient.

An isolated polypeptide or peptide of the invention can be used as an immunogen to generate antibodies using standard techniques for polyclonal and monoclonal antibody preparation. The full-length polypeptide or a functional domain of the polypeptide, either native or denatured, can be used or, alternatively, the invention provides antigenic polypeptides or peptides for use as immunogens. The antigenic peptide of a polypeptide of the invention comprises at least 8 (preferably 10, 15, 20, or 30) amino acid residues of the amino acid sequence of SEQ ID NO: 2 or a variant thereof, and features an epitope of the polypeptide such that an antibody raised against the peptide forms a specific immune complex with the polypeptide, and alternatively with a native polypeptide.

Preferred epitopes encompassed by the antigenic peptide are regions that are located on the surface of the polypeptide, e.g., hydrophilic regions. In certain embodiments, the nucleic acid molecules of the invention are present as part of nucleic acid molecules comprising nucleic acid sequences that contain or encode heterologous (e.g., vector, expression vector, or fusion polypeptide) sequences. These nucleotides can then be used to express polypeptides which can be used as immunogens to generate an immune response, or more particularly, to generate polyclonal or monoclonal antibodies specific to the expressed polypeptide.

For the production of antibodies against HBMYCNG, various host animals may be immunized by injection with the protein or a portion thereof. Such host animals include rabbits, mice, rats, and baboons. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition,” as used herein, refers to a population of antibody molecules that contain only one species of an antigen binding site capable of immunoreacting with a particular epitope.

Polyclonal antibodies are heterogeneous populations of antibody molecules derived from the sera of animals immunized with an antigen, such as a HBMYCNG polypeptide, or an antigenic functional derivative thereof. For the production of polyclonal antibodies, host animals such as those described above, may be immunized by injection with the HBMYCNG polypeptide supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridomas producing the monoclonal antibodies of this invention may be cultivated in vitro or in vivo.

In addition, techniques developed for the production of chimeric antibodies (Morrison et al., 1984, Proc. Natl. Acad. Sci., 81: 6851-6855; Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397.)

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a polypeptide or peptide of the invention can be selected for (e.g., partially purified) or purified by, e.g., affinity chromatography. For example, a recombinantly expressed and purified (or partially purified) polypeptide of the invention is produced as described herein, and covalently or non-covalently coupled to a solid support such as, for example, a chromatography column. The column can then be used to affinity purify antibodies specific for the polypeptides of the invention from a sample containing antibodies directed against a large number of different epitopes, thereby generating a substantially purified antibody composition, i.e., one that is substantially free of contaminating antibodies.

By a substantially purified antibody composition is meant, in this context, that the antibody sample contains at most only 30% (by dry weight) of contaminating antibodies directed against epitopes other than those on the desired polypeptide or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired polypeptide or peptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

In addition, techniques have been developed for the production of humanized antibodies (see, e.g., Queen, U.S. Pat. No. 5,585,089). Humanized antibodies are antibody molecules from non-human species having one or more CDRs from the non-human species and a framework region from a human immunoglobulin molecule.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) Bio/technology 12:899-903).

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be used in the production of single chain antibodies against HBMYCNG. Single chain antibodies are formed by linking the heavy and light chain fragments of the Fv region via an amino acid bridge, resulting in a single chain polypeptide.

Furthermore, antibody fragments which recognize specific epitopes of HBMYCNG may be produced by techniques well known in the art. For example, such fragments include but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity.

An antibody directed against a polypeptide of the invention (e.g., monoclonal antibody) can be used to isolate the polypeptide by standard techniques, such as affinity chromatography or immunoprecipitation. Moreover, such an antibody can be used to detect the polypeptide (e.g., in a cellular lysate or cell supernatant) in order to evaluate the abundance and pattern of expression of the polypeptide. The antibodies can also be used diagnostically to monitor polypeptide levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ⁹⁹Tc or ³H.

In addition, the HBMYCNG gene sequences and gene products, including polypeptides, peptides, fusion polypeptides or peptides, and antibodies directed against said gene products and peptides, have applications for purposes independent of the role of the gene products. For example, HBMYCNG gene products, including polypeptides or peptides, as well as specific antibodies thereto, can be used for construction of fusion polypeptides to facilitate recovery, detection, or localization of another polypeptide of interest. In addition, HBMYCNG genes and gene products can be used for genetic mapping. Finally, HBMYCNG nucleic acids and gene products have generic uses, such as supplemental sources of nucleic acids, polypeptides and amino acids for food additives or cosmetic products.

Further, an antibody of the invention (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In addition, polypeptides, agonists or antagonists which bind a polypeptide of the invention can also be conjugated to the foregoing, thereby targeting a toxin to cells expressing HGPRBMY1.

The conjugates of the invention can be used for modifying a given biological response, the drug moiety is not to be construed as limited to classical chemical therapeutic agents. For example, the drug moiety may be a polypeptide or peptide possessing a desired biological activity. Such polypeptides may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-4 (“IL-4”), interleukin-6 (“IL-6”), interleukin-7 (“IL-7”), granulocyte macrophage colony stimulating factor (“GM-CSF”), granulocyte colony stimulating factor (“G-CSF”), interleukin-10 (“IL-10”), interleukin-12 (“IL-12”), interleukin-17 (“IL-15”), interleukin-17 (“IL-17”), interferon-γ (“IFN-γ”) interferon-α (“IFN-α”), or other immune factors or growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

An antibody with or without a therapeutic moiety conjugated to it can be used as a therapeutic that is administered alone or in combination with chemotherapeutic agents.

Alternatively, an antibody of the invention can be conjugated to a second antibody to form an “antibody heteroconjugate” as described by Segal in U.S. Pat. No. 4,676,980 or alternatively, the antibodies can be conjugated to form an “antibody heteropolymer” as described in Taylor et al., in U.S. Pat. Nos. 5,470,570 and 5,487,890.

An antibody with or without a therapeutic moiety conjugated to it can be used as a therapeutic that is administered alone or in combination with cytotoxic factor(s) and/or cytokine(s).

In yet a further aspect, the invention provides substantially purified antibodies or fragments thereof, including human or non-human antibodies or fragments thereof, which antibodies or fragments specifically bind to a polypeptide of the invention comprising an amino acid sequence of SEQ ID NO: 2 or a variant thereof. In various embodiments, the substantially purified antibodies of the invention, or fragments thereof, can be human, non-human, chimeric and/or humanized antibodies.

In another aspect, the invention provides human or non-human antibodies or fragments thereof, which antibodies or fragments specifically bind to a polypeptide comprising an amino acid sequence of SEQ ID NO: 2 or a variant thereof. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide of the invention comprising an amino acid sequence of SEQ ID NO: 2 or a variant thereof. The monoclonal antibodies can be human, humanized, chimeric and/or non-human antibodies.

The substantially purified antibodies or fragments thereof specifically bind to a signal peptide, a secreted sequence, an extracellular domain, a transmembrane or a cytoplasmic domain of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence, or alternatively, to an extracellular domain of the amino acid sequence of the invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes HBMYCNG, the method comprising immunizing a mammal with a polypeptide. After immunization, a sample is collected from the mammal that contains an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

5.4. Uses of the HBMYCNG Nucleic Acid Molecules, Gene Products, and Antibodies

As discussed supra, the HBMYCNG gene of this invention encodes a protein involved in the formation or function of ion channels, more particularly, cation channels. Given the importance of cations such as calcium, sodium or potassium in many cellular processes, the HBMYCNG nucleic acid molecules and polypeptides of this invention are useful for the diagnosis and treatment of a variety of human disease conditions which involve ion, more particularly, cation, channel dysfunction.

For example, calcium plays a role in the release of neurotransmitters, hormones and other circulating factors, the expression of numerous regulatory genes as well as the cellular process of apoptosis or cell death. Potassium provides for neuroprotection and also affects insulin secretion. Sodium is involved in the regulation of normal neuronal action potential generation and propagation. Sodium channel blockers such as lidocaine are important analgesics. Therefore, cation channel dysfunction may play a role in many human diseases and disorders such as CNS disorders, e.g., stroke, anxiety, and depression, Alzheimer's disease, or Parkinson's disease, and other diseases such as cardiac disorders, e.g., arrhythmia, diabetes, chronic pain, hypercalcemia, hypercalciuria, or ion channel dysfunction that is associated with immunological disorders, gastrointestinal (GI) tract disorders, or renal or liver disease. Moreover, modulation of calcium transport may play a role in the proper functioning of the serotonin nervous system which also participates in the control of anxiety, fear, depression, sleep and pain. Accordingly, cation channel dysfunction may further play a role in anxiety and fear disorders, bipolar and major depression, panic disorder, headaches, migraine, disorders of circadian rhythmicity, stress, various sexual dysfunctions including but not limited to erectile dysfunction, neuroleptic-induced catalepsy, Rett syndrome and aggressive behaviors. As such, proteins that are involved in either the formation or function of these ion channels (and the nucleic acids that encode those proteins) are useful for the diagnosis and treatment of many human diseases.

Among the uses for the nucleic acid molecules and polypeptides of the invention are the prognostic and diagnostic evaluation of human disorders involving ion/cation channel dysfunction, and the identification of subjects with a predisposition to such disorders, as described below. Other uses include methods for the treatment of such ion/cation channel dysfunction disorders, for the modulation of HBMYCNG gene-mediated activity, and for the modulation of HBMYCNG-mediated effector functions.

In addition, the nucleic acid molecules and polypeptides of the invention can be used in assays for the identification of compounds which modulate the expression of the HBMYCNG genes of the invention and/or the activity of the HBMYCNG gene products. Such compounds can include, for example, other cellular products or small molecule compounds that are involved in cation homeostasis or activity.

5.4.1. Diagnosis and Prognosis of Ion-Related Disorders

Methods of the invention for the diagnosis and prognosis of human diseases involving ion, e.g., cation, dysfunction may utilize reagents such as the HBMYCNG nucleic acid molecules and sequences described in Sections 5.1, supra, or antibodies directed against HBMYCNG polypeptides, including peptide fragments thereof, as described in Section 5.3., supra. Specifically, such reagents may be used, for example, for: (1) the detection of the presence of HBMYCNG gene mutations, or the detection of either over- or under-expression of HBMYCNG gene mRNA relative to the non-cation dysfunctional state or the qualitative or quantitative detection of alternatively spliced forms of HBMYCNG transcripts which may correlate with certain ion homeostasis disorders or susceptibility toward such disorders; and (2) the detection of either an over- or an under-abundance of HBMYCNG gene product relative to the non-cation dysfunctional state or the presence of a modified (e.g., less than full length) HBMYCNG gene product which correlates with a cation dysfunctional state or a progression toward such a state.

The methods described herein may be performed, for example, by utilizing pre-packaged diagnostic test kits comprising at least one specific HBMYCNG gene nucleic acid or anti-HBMYCNG gene antibody reagent described herein, which may be conveniently used, e.g., in clinical settings, to screen and diagnose patients exhibiting ion/cation channel/homeostasis abnormalities and to screen and identify those individuals exhibiting a predisposition to such abnormalities.

For the detection of HBMYCNG mutations, any nucleated cell can be used as a starting source for genomic nucleic acid. For the detection of HBMYCNG transcripts or HBMYCNG gene products, any cell type or tissue in which the HBMYCNG gene is expressed may be utilized.

Nucleic acid-based detection techniques are described in Section 5.4.1.1., infra, whereas peptide-based detection techniques are described in Section 5.4.1.2., infra.

5.4.1.1. Detection of Hbmycng Gene Nucleic Acid Molecules

Mutations or polymorphisms within the HBMYCNG gene can be detected by utilizing a number of techniques. Nucleic acid from any nucleated cell can be used as the starting point for such assay techniques, and may be isolated according to standard nucleic acid preparation procedures which are well known to those of skill in the art.

Genomic DNA may be used in hybridization or amplification assays of biological samples to detect abnormalities involving HBMYCNG gene structure, including point mutations, insertions, deletions and chromosomal rearrangements. Such assays may include, but are not limited to, direct sequencing (Wong, C. et al., 1987, Nature 330:384-386), single stranded conformational polymorphism analyses (SSCP; Orita, M. et al., 1989, Proc. Natl. Acad. Sci. USA 86:2766-2770), heteroduplex analysis (Keen, T. J. et al., 1991, Genomics 11:199-205; Perry, D. J. & Carrell, R. W., 1992), denaturing gradient gel electrophoresis (DGGE; Myers, R. M. et al., 1985, Nucl. Acids Res. 13:3131-3145), chemical mismatch cleavage (Cotton, R. G. et al., 1988, Proc. Natl. Acad. Sci. USA 85:4397-4401) and oligonucleotide hybridization (Wallace, R. B. et al., 1981, Nucl. Acids Res. 9:879-894; Lipshutz, R. J. et al., 1995, Biotechniques 19:442-447).

Diagnostic methods for the detection of HBMYCNG gene specific nucleic acid molecules, in patient samples or other appropriate cell sources, may involve the amplification of specific gene sequences, e.g., by PCR, followed by the analysis of the amplified molecules using techniques well known to those of skill in the art, such as, for example, those listed above. Utilizing analysis techniques such as these, the amplified sequences can be compared to those which would be expected if the nucleic acid being amplified contained only normal copies of the HBMYCNG gene in order to determine whether a HBMYCNG gene mutation exists.

Further, well-known genotyping techniques can be performed to type polymorphisms that are in close proximity to mutations in the HBMYCNG gene itself. These polymorphisms can be used to identify individuals in families likely to carry mutations. If a polymorphism exhibits linkage disequilibrium with mutations in the HBMYCNG gene, it can also be used to identify individuals in the general population likely to carry mutations. Polymorphisms that can be used in this way include restriction fragment length polymorphisms (RFLPs), which involve sequence variations in restriction enzyme target sequences, single-base polymorphisms and simple sequence repeat polymorphisms (SSLPs).

For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the HBMYCNG gene, and the diagnosis of diseases and disorders related to HBMYCNG mutations.

Also, Caskey et al. (U.S. Pat. No. 5,364,759) describe a DNA profiling assay for detecting short tri- and tetra-nucleotide repeat sequences. The process includes extracting the DNA of interest, such as the HBMYCNG gene, amplifying the extracted DNA, and labelling the repeat sequences to form a genotypic map of the individual's DNA.

A HBMYCNG probe could additionally be used to directly identify RFLPs. Additionally, a HBMYCNG probe or primers derived from the HBMYCNG sequences of the invention could be used to isolate genomic clones such as YACs, BACs, PACs, cosmids, phage or plasmids. The DNA contained in these clones can be screened for single-base polymorphisms or simple sequence length polymorphisms (SSLPs) using standard hybridization or sequencing procedures.

Alternative diagnostic methods for the detection of HBMYCNG gene-specific mutations or polymorphisms can include hybridization techniques which involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including the HBMYCNG nucleic acid molecules of the invention including recombinant DNA molecules, cloned genes or degenerate variants thereof, as described in Section 5.1 supra, under conditions favorable for the specific annealing of these reagents to their complementary sequences within the HBMYCNG gene. Preferably, the lengths of these nucleic acid reagents are at least 15 to 30 nucleotides. After incubation, all non-annealed nucleic acids are removed from the nucleic acid:HBMYCNG molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid molecules of the invention as described in Section 5.1 are easily removed. Detection of the remaining, annealed, labeled HBMYCNG nucleic acid reagents is accomplished using standard techniques well-known to those in the art. The HBMYCNG gene sequences to which the nucleic acid molecules of the invention have annealed can be compared to the annealing pattern expected from a normal HBMYCNG gene sequence in order to determine whether a HBMYCNG gene mutation is present.

Quantitative and qualitative aspects of HBMYCNG gene expression can also be assayed. For example, RNA from a cell type or tissue known, or suspected, to express the HBMYCNG gene may be isolated and tested utilizing hybridization or PCR techniques as described supra. The isolated cells can be derived from cell culture or from a patient. The analysis of cells taken from culture may be a necessary step in the assessment of cells to be used as part of a cell-based gene therapy technique or, alternatively, to test the effect of compounds on the expression of the HBMYCNG gene. Such analyses may reveal both quantitative and qualitative aspects of the expression pattern of the HBMYCNG gene, including activation or inactivation of HBMYCNG gene expression and presence of alternatively spliced HBMYCNG transcripts.

In one embodiment of such a detection scheme, a cDNA molecule is synthesized from an RNA molecule of interest (e.g., by reverse transcription of the RNA molecule into cDNA). All or part of the resulting cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the HBMYCNG nucleic acid molecules of the invention as described in Section 5.1, supra. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Such RT-PCR techniques can be utilized to detect differences in HBMYCNG transcript size which may be due to normal or abnormal alternative splicing. Additionally, such techniques can be utilized to detect quantitative differences between levels of full length and/or alternatively spliced HBMYCNG transcripts detected in normal individuals relative to those individuals exhibiting ion dysfunction disorders or exhibiting a predisposition to toward such disorders.

In the case where detection of specific alternatively spliced species is desired, appropriate primers and/or hybridization probes can be used, such that, in the absence of such sequence, no amplification would occur. Alternatively, primer pairs may be chosen utilizing the sequences depicted in FIG. 1, 3 or 5 to choose primers which will yield fragments of differing size depending on whether a particular exon is present or absent from the HBMYCNG transcript being utilized.

As an alternative to amplification techniques, standard Northern analyses can be performed if a sufficient quantity of the appropriate cells can be obtained. Utilizing such techniques, quantitative as well as size-related differences between HBMYCNG transcripts can also be detected.

Additionally, it is possible to perform HBMYCNG gene expression assays in situ, i.e., directly upon tissue sections (fixed and/or frozen) of patient tissue obtained from biopsies or resections, such that no nucleic acid purification is necessary. The nucleic acid molecules of the invention as described in Section 5.1 may be used as probes and/or primers for such in situ procedures (see, for example, Nuovo, G. J., 1992, “PCR In Situ Hybridization: Protocols And Applications”, Raven Press, NY).

5.4.1.2. Detection of HBMYCNG Gene Products

Antibodies directed against wild type or mutant HBMYCNG gene products or conserved variants or peptide fragments or extracellular domain thereof as described supra may also be used for the diagnosis and prognosis of ion or cation-related disorders. Such diagnostic methods may be used to detect abnormalities in the level of HBMYCNG gene expression or abnormalities in the structure and/or temporal, tissue, cellular, or subcellular location of HBMYCNG gene products. Antibodies, or fragments of antibodies, may be used to screen potentially therapeutic compounds in vitro to determine their effects on HBMYCNG gene expression and HBMYCNG peptide production. The compounds which have beneficial effects on ion and cation-related disorders can be identified and a therapeutically effective dose determined.

In vitro immunoassays may be used, for example, to assess the efficacy of cell-based gene therapy for ion or cation-related disorders. For example, antibodies directed against HBMYCNG peptides may be used in vitro to determine the level of HBMYCNG gene expression achieved in cells genetically engineered to produce HBMYCNG peptides. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the HBMYCNG gene. The protein isolation methods employed may, for example, be such as those described in Harlow, E. and Lane, D., 1988, “Antibodies: A Laboratory Manual”, Cold Spring Harbor Laboratory Press, Cold Spring Harbor, N.Y. The isolated cells can be derived from cell culture or from a patient. The analysis of cells taken from culture may be a necessary step in the assessment of cells to be used as part of a cell-based gene therapy technique or, alternatively, to test the effect of compounds on the expression of the HBMYCNG gene.

Preferred diagnostic methods for the detection of HBMYCNG gene products or conserved variants or peptide fragments thereof, may involve, for example, immunoassays wherein the HBMYCNG gene products or conserved variants, including gene products which are the result of alternatively spliced transcripts, or peptide fragments are detected by their interaction with an anti-HBMYCNG gene product-specific antibody.

For example, antibodies, or fragments of antibodies, such as those described in Section 5.3 supra, may be used to quantitatively or qualitatively detect the presence of HBMYCNG gene products or conserved variants or peptide fragments thereof. The antibodies (or fragments thereof) may, additionally, be employed histologically, as in immunofluorescence or immunoelectron microscopy, for in situ detection of HBMYCNG gene products or conserved variants or peptide fragments thereof. In situ detection may be accomplished by removing a histological specimen from a patient, and applying thereto a labeled HBMYCNG antibody of the present invention. The antibody (or fragment) is preferably applied by overlaying the labeled antibody (or fragment) onto a biological sample. Through the use of such a procedure, it is possible to determine not only the presence of the HBMYCNG gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for HBMYCNG gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying HBMYCNG gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled HBMYCNG gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-HBMYCNG gene product antibody may be determined according to well known methods. Those skilled in the art will be able to determine operative and optimal assay conditions for each determination by employing routine experimentation.

One of the ways in which the HBMYCNG gene peptide-specific antibody can be detectably labeled is by linking the antibody to an enzyme in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect HBMYCNG gene peptides through the use of a radioimmunoassay (RIA) (see, for example, Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

5.4.2. Screening Assays for Compounds That Modulate HBMYCNG Activity

Screening assays can be used to identify compounds that modulate HBMYCNG activity. These compounds can include, but are not limited to, peptides, small organic or inorganic molecules or macromolecules such as nucleic acid molecules or proteins, and may be utilized, e.g., in the control of ion and cation-related disorders, in the modulation of cellular processes such as the release of neurotransmitters or other cellular regulatory factors, cell activation or regulation, cell death and changes in cell membrane properties. These compounds may also be useful, e.g., in elaborating the biological functions of HBMYCNG gene products, modulating those biological functions and for ameliorating symptoms of ion or cation-related disorders.

The compositions of the invention include pharmaceutical compositions comprising one or more of these compounds. Such pharmaceutical compositions can be formulated as discussed in Section 5.5, infra.

More specifically, these compounds can include compounds that bind to HBMYCNG gene products, compounds that bind to other proteins that interact with a HBMYCNG gene product and/or interfere with the interaction of the HBMYCNG gene product with other proteins, and compounds that modulate the activity of the HBMYCNG gene, i.e., modulate the level of HBMYCNG gene expression and/or modulate the level of HBMYCNG gene product activity.

For example, assays may be utilized that identify compounds that bind to HBMYCNG gene regulatory sequences, e.g., promoter sequences (see e.g., Platt, K. A., 1994, J. Biol. Chem. 269:28558-28562), which compounds may modulate the level of HBMYCNG gene expression. In addition, functional assays can be used to screen for compounds that modulate HBMYCNG gene product activity. In such assays, compounds are screened for agonistic or antagonistic activity with respect to a biological activity or function of the HBMYCNG gene product, such as changes in the intracellular levels of an ion or cation, changes in regulatory factor release, or other activities or functions of the HBMYCNG polypeptides of the invention.

According to a preferred embodiment, a Ca²⁺ flux assay can be utilized to monitor calcium uptake in HBMYCNG-expressing host cells. The host cells are pre-loaded with a Ca²⁺-sensitive fluorescently-labeled dye (e.g., Fluo-4, Fluo-3, Indo-1 or Fura-2), i.e., the intracellular calcium is fluorescently labelled with the dye, and the effect of the compound, e.g., on the intracellular levels of the labeled-calcium determined and compared to the intracellular levels of control cells, e.g., lacking exposure to the compound of interest. Compounds that have an agonistic, i.e., stimulatory, modulatory effect on HBMYCNG activity are those that, when contacted with the HBMYCNG-expressing cells, produce an increase in intracellular calcium relative to the control cells, whereas those compounds having an antagonistic modulatory effect on HBMYCNG activity will be those that block the effects of agonists or cyclic nucleotides that increase channel activity. A Ca²⁺ flux assay is exemplified in Example Section 6.1, infra.

Functional assays for monitoring the effects of compounds on the levels or flux of other ions can be similarly performed; for example, the levels of potassium can be monitored using rubidium influx.

Screening assays may also be designed to identify compounds capable of binding to the HBMYCNG gene products of the invention. Such compounds may be useful, e.g., in modulating the activity of wild type and/or mutant HBMYCNG gene products, in elaborating the biological function of the HBMYCNG gene product, and in screens for identifying compounds that disrupt normal HBMYCNG gene product interactions, or may in themselves disrupt such interactions.

The principle of such screening assays to identify compounds that bind to the HBMYCNG gene product involves preparing a reaction mixture of the HBMYCNG gene product and the test compound under conditions and for a time sufficient to allow the two components to interact with, i.e., bind to, and thus form a complex, which can represent a transient complex, which can be removed and/or detected in the reaction mixture. For example, one assay involves anchoring a HBMYCNG gene product or the test substance onto a solid phase and detecting HBMYCNG gene product/test compound complexes anchored on the solid phase at the end of the reaction. In one embodiment of such a method, the HBMYCNG gene product may be anchored onto a solid surface, and the test compound, which is not anchored, may be labeled, either directly or indirectly.

The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for HBMYCNG gene product or the test compound to anchor any complexes formed in solution, and a labeled antibody specific for the other component of the possible complex to detect anchored complexes.

Compounds that modulate HBMYCNG gene product activity can also include compounds that bind to proteins that interact with the HBMYCNG gene product. These modulatory compounds can be identified by first identifying those proteins that interact with the HBMYCNG gene product, e.g., by standard techniques known in the art for detecting protein-protein interactions, such as co-immunoprecipitation, crosslinking and co-purification through gradients or chromatographic columns. Utilizing procedures such as these allows for the isolation of proteins that interact with HBMYCNG gene products or polypeptides of the invention as described supra.

Once isolated, such a protein can be identified and can, in turn, be used, in conjunction with standard techniques, to identify additional proteins with which it interacts. For example, at least a portion of the amino acid sequence of the protein that interacts with the HBMYCNG gene product can be ascertained using techniques well known to those of skill in the art, such as via the Edman degradation technique (see, e.g., Creighton, 1983, “Proteins: Structures and Molecular Principles”, W. H. Freeman & Co., N.Y., pp.34-49). The amino acid sequence thus obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and screening are well-known (see, e.g., Ausubel, supra., and PCR Protocols: A Guide to Methods and Applications, 1990, Innis, M. et al., eds. Academic Press, Inc., New York).

Additionally, methods may be employed that result in the simultaneous identification of genes which encode proteins interacting with HBMYCNG gene products or polypeptides. These methods include, for example, probing expression libraries with labeled HBMYCNG protein, using HBMYCNG protein in a manner similar to the well known technique of antibody probing of λgt11 libraries. One method that detects protein interactions in vivo is the two-hybrid system. A version of this system in described by Chien et al., 1991, Proc. Natl. Acad. Sci. USA, 88:9578-9582 and is commercially available from Clontech (Palo Alto, Calif.).

In addition, compounds that disrupt HBMYCNG interactions with its interacting or binding partners, as determined immediately above, may be useful in regulating the activity of the HBMYCNG gene product, including mutant HBMYCNG gene products. Such compounds may include, but are not limited to molecules such as peptides, and the like, which may bind to the HBMYCNG gene product as described above.

The basic principle of the assay systems used to identify compounds that interfere with the interaction between the HBMYCNG gene product and its interacting partner or partners involves preparing a reaction mixture containing the HBMYCNG gene product, and the interacting partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of HBMYCNG gene product and its interacting partner. Control reaction mixtures are incubated without the test compound or with a placebo. The formation of any complexes between the HBMYCNG gene product and the interacting partner is then detected. The formation of a complex in the control reaction, but not in the reaction mixture containing the test compound, indicates that the compound interferes with the interaction of the HBMYCNG gene product and the interacting partner. Additionally, complex formation within reaction mixtures containing the test compound and a normal HBMYCNG gene product may also be compared to complex formation within reaction mixtures containing the test compound and a mutant HBMYCNG gene product. This comparison may be important in those cases wherein it is desirable to identify compounds that disrupt interactions of mutant but not normal HBMYCNG proteins.

The assay for compounds that interfere with the interaction of HBMYCNG gene products and interacting partners can be conducted in a heterogeneous or homogeneous format. Heterogeneous assays involve anchoring either the HBMYCNG gene product or the binding partner onto a solid phase and detecting complexes anchored on the solid phase at the end of the reaction.

In homogeneous assays, the entire reaction is carried out in a liquid phase. In either approach, the order of addition of reactants can be varied to obtain different information about the compounds being tested. For example, test compounds that interfere with the interaction between the HBMYCNG gene products and the interacting partners, e.g., by competition, can be identified by conducting the reaction in the presence of the test substance; i.e., by adding the test substance to the reaction mixture prior to or simultaneously with the HBMYCNG gene product and interacting partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the HBMYCNG gene product or the interacting partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the HBMYCNG gene product or interacting partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the interacting components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified.

In an alternate embodiment, a preformed complex of the HBMYCNG gene protein and the interacting partner is prepared in which either the HBMYCNG gene product or its interacting partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt HBMYCNG gene protein/interacting partner interaction can be identified.

In another embodiment of the invention, these same techniques can be employed using peptide fragments that correspond to the binding domains of the HBMYCNG protein and/or the interacting partner, in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interacting, e.g., binding. Alternatively, one protein can be anchored to a solid surface using methods described in this Section above, and allowed to interact with, e.g., bind, to its labeled interacting partner, which has been treated with a proteolytic enzyme, such as trypsin. After washing, a short, labeled peptide comprising the interacting, e.g., binding, domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

The human HBMYCNG polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HBMYCNG polypeptide, or a bindable peptide fragment, of this invention, comprising providing a plurality of compounds, combining the HBMYCNG polypeptide, or a bindable peptide fragment, with each of a plurality of compounds for a time sufficient to allow binding under suitable conditions and detecting binding of the HBMYCNG polypeptide or peptide to each of the plurality of test compounds, thereby identifying the compounds that specifically bind to the HBMYCNG polypeptide or peptide.

Methods of identifying compounds that modulate the activity of the novel human HBMYCNG polypeptides and/or peptides are provided by the present invention and comprise combining a potential or candidate compound or drug modulator of ion channel biological activity with an HBMYCNG polypeptide or peptide, for example, the HBMYCNG amino acid sequence as set forth in SEQ ID NOS:2, and measuring an effect of the candidate compound or drug modulator on the biological activity of the HBMYCNG polypeptide or peptide. Such measurable effects include, for example, physical binding interaction; the ability to cleave a suitable ion channel substrate; effects on native and cloned HBMYCNG-expressing cell line; and effects of modulators or other ion channel-mediated physiological measures.

Another method of identifying compounds that modulate the biological activity of the novel HBMYCNG polypeptides of the present invention comprises combining a potential or candidate compound or drug modulator of a ion channel biological activity with a host cell that expresses the HBMYCNG polypeptide and measuring an effect of the candidate compound or drug modulator on the biological activity of the HBMYCNG polypeptide. The host cell can also be capable of being induced to express the HBMYCNG polypeptide, e.g., via inducible expression. Physiological effects of a given modulator candidate on the HBMYCNG polypeptide can also be measured. Thus, cellular assays for particular ion channel modulators may be either direct measurement or quantification of the physical biological activity of the HBMYCNG polypeptide, or they may be measurement or quantification of a physiological effect. Such methods preferably employ a HBMYCNG polypeptide as described herein, or an overexpressed recombinant HBMYCNG polypeptide in suitable host cells containing an expression vector as described herein, wherein the HBMYCNG polypeptide is expressed, overexpressed, or undergoes upregulated expression.

Another aspect of the present invention embraces a method of screening for a compound that is capable of modulating the biological activity of a HBMYCNG polypeptide, comprising providing a host cell containing an expression vector harboring a nucleic acid sequence encoding a HBMYCNG polypeptide, or a functional peptide or portion thereof (e.g., SEQ ID NOS:2); determining the biological activity of the expressed HBMYCNG polypeptide in the absence of a modulator compound; contacting the cell with the modulator compound and determining the biological activity of the expressed HBMYCNG polypeptide in the presence of the modulator compound. In such a method, a difference between the activity of the HBMYCNG polypeptide in the presence of the modulator compound and in the absence of the modulator compound indicates a modulating effect of the compound.

Essentially any chemical compound can be employed as a potential modulator or ligand in the assays according to the present invention. Compounds tested as ion channel modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel HBMYCNG polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HBMYCNG polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HBMYCNG polypeptide or peptide to measure a biological binding or ligand binding activity, the source may be a whole cell lysate that can be prepared by successive freeze-thaw cycles (e.g., one to three) in the presence of standard protease inhibitors. The HBMYCNG polypeptide may be partially or completely purified by standard protein purification methods, e.g., affinity chromatography using specific antibody described infra, or by ligands specific for an epitope tag engineered into the recombinant HBMYCNG polypeptide molecule, also as described herein. Binding activity can then be measured as described.

Compounds which are identified according to the methods provided herein, and which modulate or regulate the biological activity or physiology of the HBMYCNG polypeptides according to the present invention are a preferred embodiment of this invention. It is contemplated that such modulatory compounds may be employed in treatment and therapeutic methods for treating a condition that is mediated by the novel HBMYCNG polypeptides by administering to an individual in need of such treatment a therapeutically effective amount of the compound identified by the methods described herein.

In addition, the present invention provides methods for treating an individual in need of such treatment for a disease, disorder, or condition that is mediated by the HBMYCNG polypeptides of the invention, comprising administering to the individual a therapeutically effective amount of the HBMYCNG-modulating compound identified by a method provided herein.

5.4.3. Methods and Compositions for the Treatment of Ion Channel-Related Disorders

The present invention also relates to methods and compositions for the treatment or modulation of any disorder or cellular process that is mediated or regulated by HBMYCNG gene product expression or function, e.g., HBMYCNG-mediated cell activation, signal transduction, cellular regulatory factor release, etc. Further, HBMYCNG effector functions can be modulated via such methods and compositions.

The methods of the invention include methods that modulate HBMYCNG gene and gene product activity. In certain instances, the treatment will require an increase, upregulation or activation of HBMYCNG activity, while in other instances, the treatment will require a decrease, downregulation or suppression of HBMYCNG activity. “Increase” and “decrease” refer to the differential level of HBMYCNG activity relative to HBMYCNG activity in the cell type of interest in the absence of modulatory treatment. Methods for the decrease of HBMYCNG activity are discussed in Section 5.4.3.1, infra. Methods for the increase of HBMYCNG activity are discussed in Section 5.4.3.2, infra. Methods which can either increase or decrease HBMYCNG activity depending on the particular manner in which the method is practiced are discussed in Section 5.4.3.3, infra.

5.4.3.1. Methods for Decreasing HBMYCNG Activity

Successful treatment of ion channel/ionic homeostasis disorders, e.g., CNS disorders, cardiac disorders or hypercalcemia, can be brought about by methods which serve to decrease HBMYCNG activity. Activity can be decreased by, e.g., directly decreasing HBMYCNG gene product activity and/or by decreasing the level of HBMYCNG gene expression.

For example, compounds such as those identified through assays described in Section 5.4.2., supra, that decrease HBMYCNG gene product activity can be used in accordance with the invention to ameliorate symptoms associated with ion channel/ionic homeostasis disorders. As discussed supra, such molecules can include, but are not limited to peptides, including soluble peptides, and small organic or inorganic molecules, and can be referred to as HBMYCNG antagonists. Techniques for the determination of effective doses and administration of such compounds are described in Section 5.5., infra.

In addition, antisense and ribozyme molecules that inhibit HBMYCNG gene expression can also be used to reduce the level of HBMYCNG gene expression, thus effectively reducing the level of HBMYCNG gene product present, thereby decreasing the level of HBMYCNG activity. Still further, triple helix molecules can be utilized in reducing the level of HBMYCNG gene expression. Such molecules can be designed to reduce or inhibit either wild type, or if appropriate, mutant target gene activity. Techniques for the production and use of such molecules are well known to those of skill in the art.

Antisense approaches involve the design of oligonucleotides (either DNA or RNA) that are complementary to HBMYCNG gene mRNA. The antisense oligonucleotides will bind to the complementary HBMYCNG gene mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335. Thus, oligonucleotides complementary to either the 5′- or 3′-non-translated, non-coding regions of the HBMYCNG gene, as depicted in FIG. 1 could be used in an antisense approach to inhibit translation of endogenous HBMYCNG gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of target or pathway gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and non-specific biological effects of oligonucleotides. It is also preferred that these studies compare levels of the target RNA or protein with that of an internal control RNA or protein. Additionally, results obtained using the antisense oligonucleotide are preferably compared with those obtained using a control oligonucleotide. It is preferred that the control oligonucleotide is of approximately the same length as the antisense oligonucleotide and that the nucleotide sequence of the control oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc.

The oligonucleotide may also include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Application No. WO 88/09810) or the blood-brain barrier (see, e.g., PCT Application No. WO 89/10134), or hybridization-triggered cleavage agents (see, e.g., Krol et al., 1988, BioTechniques 6:958-976) or intercalating agents (see, e.g., Zon, 1988, Pharm. Res. 5:539-549). For example, the oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209) and methylphosphonate oligonucleotides can be prepared by use of controlled pore glass polymer supports (Sarin et al., 1988, Proc. Natl. Acad. Sci. U.S.A. 85:7448-7451), etc.

The antisense molecules should be delivered to cells which express the HBMYCNG gene in vivo. A number of methods have been developed for delivering antisense DNA or RNA to cells; e.g., antisense molecules can be injected directly into the tissue site or modified antisense molecules designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Thus, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous HBMYCNG gene transcripts and thereby prevent translation of the HBMYCNG gene mRNA. For example, a vector can be introduced in vivo such that it is taken up by a cell and directs the transcription of an antisense RNA.

Ribozymes are enzymatic RNA molecules capable of catalyzing the specific cleavage of RNA (For a review, see, e.g., Rossi, J., 1994, Current Biology 4:469-471). The mechanism of ribozyme action involves sequence-specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave HBMYCNG gene mRNA transcripts can also be used to prevent translation of HBMYCNG gene mRNA and expression of target or pathway genes. (See, e.g., PCT Application No. WO 90/11364; Sarver et al., 1990, Science 247:1222-1225).

The ribozymes of the present invention also include RNA endoribonucleases (hereinafter referred to as “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; PCT Patent Application No. WO 88/04300; Been and Cech, 1986, Cell, 47:207-216). The Cech-type ribozymes have an eight base pair active site which hybridizes to a target RNA sequence, after which cleavage of the target RNA takes place. The invention encompasses those Cech-type ribozymes which target eight base-pair active site sequences that are present in an HBMYCNG gene.

As in the antisense approach, the ribozymes can be composed of modified oligonucleotides (e.g. for improved stability, targeting, etc.) and should be delivered to cells which express the HBMYCNG gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous HBMYCNG gene messages and inhibit translation. Because ribozymes, unlike antisense molecules, are catalytic, a lower intracellular concentration is required for efficiency.

Endogenous HBMYCNG gene expression can also be reduced by inactivating or “knocking out” the target and/or pathway gene or its promoter using targeted homologous recombination (see, e.g., Smithies et al., 1985, Nature 317:230-234; Thomas & Capecchi, 1987, Cell 51:503-512; Thompson et al., 1989 Cell 5:313-321). For example, a mutant, non-functional HBMYCNG gene (or a completely unrelated DNA sequence) flanked by DNA homologous to the endogenous HBMYCNG gene (either the coding regions or regulatory regions of the HBMYCNG gene) can be used, with or without a selectable marker and/or a negative selectable marker, to transfect cells that express the HBMYCNG gene in vivo. Insertion of the DNA construct, via targeted homologous recombination, results in inactivation of the HBMYCNG gene. Such techniques can also be utilized to generate ion/cation disorder animal models. It should be noted that this approach can be adapted for use in humans provided the recombinant DNA constructs are directly administered or targeted to the required site in vivo using appropriate viral vectors, e.g., herpes virus vectors.

Alternatively, endogenous HBMYCNG gene expression can be reduced by targeting deoxyribonucleotide sequences complementary to the regulatory region of the HBMYCNG gene (i.e., the HBMYCNG gene promoter and/or enhancers) to form triple helical structures that prevent transcription of the HBMYCNG gene in target cells in the body (see generally, Helene, C., 1991, Anticancer Drug Des. 6(6):569-84; Helene, C., et al., 1992, Ann. N.Y. Acad. Sci. 660:27-36; and Maher, L. J., 1992, Bioassays 14(12):807-15).

Nucleic acid molecules to be used in triple helix formation for the inhibition of transcription should be single stranded and composed of deoxynucleotides. The base composition of these oligonucleotides should be designed to promote triple helix formation via Hoogsteen base pairing rules, which generally require sizeable stretches of either purines or pyrimidines to be present on one strand of the duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands of the triplex.

Alternatively, the potential sequences that can be targeted for triple helix formation may be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of the duplex.

In instances wherein the antisense, ribozyme, and/or triple helix molecules described herein are utilized to inhibit mutant HBMYCNG gene expression, it is possible that the technique may so efficiently reduce or inhibit the transcription (triple helix) and/or translation (antisense, ribozyme) of mRNA produced by normal target gene alleles that the concentration of normal target gene product present may be lower than is necessary for a normal phenotype. In such cases, to ensure that substantially normal levels of HBMYCNG gene activity are maintained, nucleic acid molecules that encode and express HBMYCNG gene polypeptides exhibiting normal target gene activity can be introduced into cells via gene therapy methods that do not contain sequences susceptible to whatever antisense, ribozyme, or triple helix treatments are being utilized. In instances where the target gene encodes an extracellular protein, it can be preferable to coadminister normal target gene protein in order to maintain the requisite level of target gene activity.

Antisense RNA and DNA, ribozyme, and triple helix molecules of the invention can be prepared by any method known in the art, e.g., methods for chemically synthesizing oligodeoxyribonucleotides and oligoribonucleotides well known in the art such as solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In addition, well-known modifications to DNA molecules can be introduced into the HBMYCNG nucleic acid molecules of the invention as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

5.4.3.2. Methods for Increasing HBMYCNG Activity

Successful treatment of ion/cation disorders can also be brought about by techniques which serve to increase the level of HBMYCNG activity. Activity can be increased by, for example, directly increasing HBMYCNG gene product activity and/or by increasing the level of HBMYCNG gene expression.

For example, compounds such as those identified through the assays described in Section 5.4.2., supra, that increase HBMYCNG activity can be used to treat ion/cation-related disorders. Such molecules can include, but are not limited to peptides, including soluble peptides, and small organic or inorganic molecules, and can be referred to as HBMYCNG agonists.

For example, a compound can, at a level sufficient to treat ion/cation-related disorders and symptoms, be administered to a patient exhibiting such symptoms. One of skill in the art will readily know how to determine the concentration of effective, non-toxic doses of the compound, utilizing techniques such as those described infra.

Alternatively, in instances wherein the compound to be administered is a peptide compound, DNA sequences encoding the peptide compound can be directly administered to a patient exhibiting an ion/cation-related disorder or symptoms, at a concentration sufficient to produce a level of peptide compound sufficient to ameliorate the symptoms of the disorder. Any of the techniques discussed infra, which achieve intracellular administration of compounds, such as, for example, liposome administration, can be utilized for the administration of such DNA molecules. In the case of peptide compounds which act extracellularly, the DNA molecules encoding such peptides can be taken up and expressed by any cell type, so long as a sufficient circulating concentration of peptide results for the elicitation of a reduction in the ion/cation disorder symptoms.

In cases where the ion/cation disorder can be localized to a particular portion or region of the body, the DNA molecules encoding such modulatory peptides may be administered as part of a delivery complex. Such a delivery complex can comprise an appropriate nucleic acid molecule and a targeting means. Such targeting means can comprise, for example, sterols lipids, viruses or target cell specific binding agents. Viral vectors can include, but are not limited to adenovirus, adeno-associated virus, and retrovirus vectors, in addition to other particles that introduce DNA into cells, such as liposomes.

Further, in instances wherein the ion/cation-related disorder involves an aberrant HBMYCNG gene, patients can be treated by gene replacement therapy. One or more copies of a normal HBMYCNG gene or a portion of the gene that directs the production of a normal HBMYCNG gene protein with HBMYCNG gene function, can be inserted into cells, via, for example a delivery complex as described supra.

Such gene replacement techniques can be accomplished either in vivo or in vitro. Techniques which select for expression within the cell type of interest are preferred. For in vivo applications, such techniques can, for example, include appropriate local administration of HBMYCNG gene sequences.

Additional methods which may be utilized to increase the overall level of HBMYCNG activity include the introduction of appropriate HBMYCNG gene-expressing cells, preferably autologous cells, into a patient at positions and in numbers which are sufficient to ameliorate the symptoms of the ion/cation-related disorder. Such cells may be either recombinant or non-recombinant. Among the cells which can be administered to increase the overall level of HBMYCNG gene expression in a patient are normal cells, which express the HBMYCNG gene. The cells can be administered at the anatomical site of expression, or as part of a tissue graft located at a different site in the body. Such cell-based gene therapy techniques are well known to those skilled in the art (see, e.g., Anderson, et al., U.S. Pat. No. 5,399,349; Mulligan and Wilson, U.S. Pat. No. 5,460,959).

HBMYCNG gene sequences can also be introduced into autologous cells in vitro. These cells expressing the HBMYCNG gene sequence can then be reintroduced, preferably by intravenous administration, into the patient until the disorder is treated and symptoms of the disorder are ameliorated.

5.4.3.3. Additional Modulatory Techniques

The present invention also includes modulatory techniques which, depending on the specific application for which they are utilized, can yield either an increase or a decrease in HBMYCNG activity levels leading to the amelioration of ion/cation-related disorders such as those described above.

Antibodies exhibiting modulatory capability can be utilized according to the methods of this invention to treat the ion/cation-related disorders. Depending on the specific antibody, the modulatory effect can be an increase or decrease in HBMYCNG activity. Such antibodies can be generated using standard techniques described in Section 5.3, supra, against full length wild type or mutant HBMYCNG proteins, or against peptides corresponding to portions of the proteins, as wells as against extracellular domains of the HBMYCNG polypeptide or HBMYCNG epitopes within the water soluble fusion protein mimic of the HMBYCNG disclosed above. The antibodies include but are not limited to polyclonal, monoclonal, Fab fragments, single chain antibodies, chimeric antibodies, etc.

Lipofectin or liposomes can be used to deliver the antibody or a fragment of the Fab region which binds to the HBMYCNG gene product epitope to cells expressing the gene product. Where fragments of the antibody are used, the smallest inhibitory fragment which binds to the HBMYCNG protein's binding domain is preferred. For example, peptides having an amino acid sequence corresponding to the domain of the variable region of the antibody that binds to the HBMYCNG protein can be used. Such peptides can be synthesized chemically or produced via recombinant DNA technology using methods well known in the art (e.g., see Creighton, 1983, supra and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al., 1993, Proc. Natl. Acad. Sci. USA 90:7889-7893.

5.5. Pharmaceutical Preparations and Methods of Administration

The compounds, e.g., nucleic acid sequences, polypeptides, peptides, and recombinant cells, described supra can be administered to a patient at therapeutically effective doses to treat or ameliorate ion/cation-related disorders. A therapeutically effective dose refers to that amount of a compound or cell population sufficient to result in amelioration of the disorder symptoms, or alternatively, to that amount of a nucleic acid sequence sufficient to express a concentration of HBMYCNG gene product which results in the amelioration of the disorder symptoms.

Toxicity and therapeutic efficacy of compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the methods of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that HBMYCNG-expressing cells be introduced into patients via intravenous administration.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

### 6. EXAMPLE

### Identification of Two Novel HBMYCNG Genes and Their Encoded Proteins

The section below describes the identification of a novel human CNG gene sequence encoding the full-length, novel human ion channel, HBMYCNG.

6.1. Cloning of Novel HBMYCNG DNA Sequences

In general all routine molecular biology procedures followed standard protocols or relied on widely available commercial kits and reagents. All sequencing was done with an ABI 373 automated sequencer using commercial dye-terminator chemistry.

Cyclic nucleotide gated channel sequences from rat, mouse and chicken were used as sequence probes in a homology search (gapped BLAST) of public domain expressed sequence tag (EST) and human genomic databases. The top EST and genomic hits from the BLAST search, (i.e. those BLAST hits whose Expectation values were less than 0.001 were selected as potential hits and selected for subsequent analysis) were used as probes in a second homology search against the non-redundant protein and patent sequence databases. The results of the second search revealed putative genomic exons which could encode a novel CNG ion channel, within Bacterial Artificial Chromosome (BAC), Accession No. AF002992.

The cDNA complete coding sequence of the HBMYCNG gene was cloned as follows. Using the predict full length sequence The following PCR Primers were designed.

| | |
|---|---|
| HuCNG2-s | |
| GCTCTAGATGTACATGGAGGATGACCGAAA | Xba 1 site |
| HuCNG2-1a | |
| CAGCCAACGCAGTCTGTACTCT | no sites, use |
| | nested primer 2 |
| HuCNG2-2a | |
| CGGGATCCGAGGCGGAATCTTGGATGTTT | BamH1 site |

Using huCNG2-s and huCNG2-1a, PCR was carried out on brain first strand cDNA made by standard techniques. To increase the specificity of the amplification, a 1 microliter aliquot was removed after the PCR reaction was complete and re-amplified using huCNG2-s and huCNG2-2a. The PCR reaction was passed over a s-400 spun-column (Amersham Pharmacia Biotech, Piscataway, N.J.) to remove excess PCR primers and DNA was digested with the restriction endonucleases Xba I and Bam HI. This reaction was extracted with phenol:chloroform and the aqueous layer precipitated with 100% ethanol and 0.3 M Sodium Acetate. The precipitated DNA was run on an 0.8% agarose gel and the DNA band purified using a QIAquick Gel extraction kit (Qiagen, Valencia Calif.). The resulting DNA was ligated to pBS-SK digested with Xba I and BamHI (Stratagene, La Jolla, Calif.)and introduced into E. coli strain DH10B using standard techniques. Positive clones were identified by PCR, using the same primers used for cloning, and several clones were sequenced using the PCR primers as well as with internal primers designed from the predicted gene sequence.

| | | | |
|---|---|---|---|
| CNG2-3s | AGAGCCTGCTTCAGTGA | 17 | Sequencing primer |
| CNG2-3a | TCACTGAAGCAGGCTCT | 17 | Sequencing primer |
| CNG2-4s | TTACTGGTCCACACTGA | 17 | Sequencing primer |
| CNG2-4a | TCAGTGTGGACCAGTAA | 17 | Sequencing primer |
| CNG2-5s | ACGCACAGCTAATATCCGCA | 20 | Sequencing primer |
| CNG2-5a | TGCGGATATTAGCTGTGCGT | 20 | Sequencing primer |

The resulting sequence was compared to the predicted sequence for completeness.

The DNA sequence for HBMYCNG is depicted in FIG. 1. The derived protein, i.e., the full-length amino acid sequence encoded by the HBMYCNG gene is depicted in FIG. 2. Analysis of the amino acid sequence of FIG. 2 for the detection of transmembrane segments was performed using the computer program TMPRED and transmembrane prediction information from related proteins. Putative transmembrane segments are depicted in bold in FIG. 3, while the predicted ion pore, located between the fifth and sixth transmembrane, counting from the amino-terminus of the protein, is underlined.

The complete sequence for HBMYCNG can be identified in a set of sequences from a large genomic fragment (AF002992) reported as part of the human genome sequencing project. The complete cDNA nucleotide sequence encoding the HBMYCNG polypeptide described herein was only partially identified in the annotations to the AF002992 BAC sequence.

6.2. Calcium Flux Assays Using the HBMYCNG Gene

Ca²⁺ flux assays are performed to determine the effect on HBMYCNG of various ligands known to affect cation channel proteins. More specifically, Ca²⁺ uptake is measured in transiently transfected CHO cells, i.e., transfected with the HBMYCNG nucleic acid molecules of the invention, using the Ca²⁺-sensitive dye Fluo-4 (Molecular Probes) in a Molecular Devices Fluorometric Imaging Plate Reader (FLIPR). Cells are loaded with the dye for 30-90 minutes prior to the experiment in the presence of sulfinpyrazone. Test reagents are added, and Ca²⁺ uptake measured over a three minute period.

Ca²⁺-flux assays may also be performed for the detection and evaluation of compounds that modulate the activity of G-protein coupled receptors. In such assays, cells expressing a G-protein coupled receptor of interest are loaded with the dye for 30-90 minutes prior to the experiment in the presence of sulfinpyrazone. Test reagents, which include test compounds, which may be agonists or antagonists of the G-protein coupled receptor are added, and Ca²⁺ uptake, reflecting the intracellular cyclic nucleotide concentration, is measured over a three minute period. In addition, these same assay techniques can be applied to other cations that enter cells through CNG channels, using appropriate dyes and incubations.

6.3. Expression Profile of HBMYCNG

The expression profile of HBMYCNG in various tissues was determined by measuring the relative abundance of HBMYCNG RNA in those tissues using quantitative PCR analyses.

Methods

Total RNA from tissues was isolated using the TriZol protocol (Invitrogen, Carlsbad, Calif.) and quantified by determining absorbance at 260 nM. An assessment of the 18S and 28S ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using ABI Primer Express software (Applied Biosystems, Foster City, Calif.)and used to amplify small amplicons (150 base pairs or less) to maximize the likelihood that the primers would function at 100% efficiency. The primer and probe sequences were searched against Public Genbank databases to ensure target specificity. Primers and probes were obtained from ABI.

For HBMYCNG the primer and probe sequences used were:

| | |
|---|---|
| Forward Primer | 5′-TCAGAGAATGGGCCAACAAGA-3′ |
| Reverse Primer | 5′-CGAAAACGCTCGAGGAATGA-3′ |
| Probe | CAGGCCTAGGTTCCTCCTCTCGGAAA |

DNA Contamination

To assess the level of contaminating genomic DNA in the RNA, the RNA was divided into 2 aliquots and one half was treated with Rnase-free Dnase (Invitrogen, Carlsbad, Calif.). RNA from both the Dnase-treated and non-treated samples were then subjected to reverse transcription reactions with (RT+) and without (RT−) the presence of reverse transcriptase. TaqMan™ assays were carried out with the gene-specific primers (see below) and the contribution of genomic DNA to the signal detected was evaluated by comparing the threshold cycles obtained with the RT+/RT− non-Dnase treated RNA to that on the RT+/RT− Dnase treated RNA. For the RNA samples used for the determination of relative expression levels, the amount of signal contributed by genomic DNA in the Dnased RT− RNA was less that 10% of that obtained with Dnased RT+ RNA.

Reverse Transcription reaction and Sequence Detection

100 ng of Dnase-treated total RNA was annealed to 2.5 mM of the gene-specific reverse primer in the presence of 5.5 mM MgCl₂ by heating the sample to 72° C. for 2 min and then cooling to 55° C. for 30 min. 1.25 U/ml of MuLv reverse transcriptase and 500 mM of each dNTP were then added to the reaction and the sample was incubated at 37° C. for 30 min. The sample was then heated to 90° C. for 5 min to denature enzyme.

Quantitative sequence detection was carried out on a ABI PRISM 7700 by adding the following components to the reverse transcribed reaction: forward and reverse primers (each to a concentration of 2.5 mM), all four dNTPs (500 mM each), buffer and 5U AmpliTaq Gold™. The PCR reaction is then held at 94° C. for 12 min, followed by 40 amplification cycles of 94° C. for 15 sec and 60° C. for 30 sec.

Data Analysis

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}. The threshold cycles for testis, raphe nucleus, and pineal gland were 32, 36.5, and 37.5, respectively, indicating that the number of copies of HBMYCNG mRNA in these samples was very low.

Results

The data obtained indicated that the HBMYCNG gene is expressed only in certain tissues and only at very low levels in those tissues. More specifically, expression of the HBMYCNG gene is 250-fold greater in testis, 10-fold greater in the raphe nucleus of the brain, and 5-fold greater in the pineal gland than in control tissues.

6.4. HBMYCNG Fusion Proteins

Chimeric proteins comprising all or a portion of the HBMYCNG protein, as depicted in FIG. 2, fused to all or a portion of a heterologous protein, are provided using recombinant DNA methods and reagents well known in the art. In specific embodiments, one or more portions of the HBMYCNG protein are fused to a portion of an immunoglobulin protein, and, more particularly, to a portion of a human IgG comprising the hinge, CH2, and CH3 regions thereof.

Such portions of the HBMYCNG protein can include, but are not limited to, one more of the extracellular domains of the HBMYCNG protein, comprising, approximately, amino acid residues 161 to 173, amino acid residues 237 to 274, and amino acid residues 370 to 453 of SEQ ID No. 2. In other embodiments, the portion of the HBMYCNG protein incorporated into a fusion includes all or a portion of the amino terminal domain of the HBMYCNG protein, comprising, approximately, amino acid residues 1 or 2 to residue 140 SEQ ID No. 2, or of the carboxy-terminal domain of the HBMYCNG protein, comprising, approximately amino acid residues 474 to 644 of SEQ ID No. 2.

DNA encoding the desired portion of the HBMYCNG protein can be isolated by PCR amplification of appropriate sequences, using, for example, cDNA as template, preferably cloned cDNA comprising the nucleotide sequence of SEQ ID NO.: 1, and appropriate upstream and downstream primers. The design, synthesis, and use of such primers are well known in the art and will include, as needed or desired, appropriate recognition sequences for one or more restriction enzymes to enable directional, in-frame cloning of a DNA fragment encoding a particular portion of the HBMYCNG protein into an expression vector in operable association with appropriate genetic expression and regulatory elements and with a second DNA sequence encoding the protein or portion thereof to which the HBMYCNG protein portion is to be fused. Examples of systems useful for the expression of such fusion proteins, in which the HBMYCNG protein portion may be positioned at either the amino-terminus, carboxyl-terminus or within a chimeric fusion protein, are disclosed supra. HBMYCNG-immunoglobulin C gamma (IgCγ) fusion proteins are prepared as described by Linsley et al., in J. Exp. Med.173:721-730 (1991), which is hereby incorporated by reference in its entirety, incorporated by reference herein. DNA encoding amino acid sequences corresponding to the desired portion of the HBMYCNG protein are joined to DNA encoding amino acid sequences corresponding to the hinge, CH2 and CH3 regions of human IgCγ1. This is accomplished using PCR amplification to generate DNA fragments encoding appropriate portions of the HBMYCNG and IgCγ proteins. PCR reactions (0.1 ml final volume) are run in Taq polymerase buffer(Stratagene, La Jolla, Calif.), containing 20μ moles each of dNTP; 50-100 pmoles of the appropriate primers; template (1 ng plasmid or cDNA synthesized as described by Kawasaki in PCR Protocols, Academic Press, pp. 21-27 (1990),incorporated by reference herein); and Taq polymerase (Stratagene). Reactions are run on a thermocycler (Perkin Elmer Corp., Norwalk, Conn.)for 16-30 cycles (a typical cycle consists of steps of 1 min at 94° C., 1-2 min at 50° C. and 1-3 min at 72° C.). Products of the PCR reactions are cleaved with appropriate restriction endonucleases at sites introduced in the PCR primers, and then are gel purified.

The 3′ portion of the fusion constructs corresponding to human IgCγ1 sequences is was made by a coupled reverse transcriptase (from Avian myeloblastosis virus; Life Sciences Associates, Bayport, N.Y.)—PCR reaction using RNA from a myeloma cell line producing human-mouse chimeric mAb L6 (available from Dr. P. Fell and M. Gayle, Bristol-Myers Squibb Company, Pharmaceutical Research Institute, Seattle, Wash.) as template. Appropriate upstream and downstream oligonucleotide, such as those described in U.S. Pat. No. 6,090,914,which is hereby incorporated by reference in its entirety, are used to amplify and isolate the desired IgCγ coding region.

Reaction products are cleaved with appropriate restriction endonucleases and gel purified. Final constructs are assembled by ligating the endonucleases cleaved fragments containing HBMYCNG sequence together with a cleaved fragment containing IgCγ1 sequences into an expression vector such as CDMB, as described in U.S. Pat. No. 6,090,917. Ligation products are transformed into MC1061/p3 E. coli cells and colonies are screened for the appropriate plasmids. Sequences of the resulting constructs are confirmed by DNA sequencing. In a preferred embodiment the HBMYCNG portion coding sequence is fused in this manner to DNA encoding amino acids corresponding to the IgCγ1 hinge region.

Cell Culture and Transfections

COS (monkey kidney cells) are transfected with expression these chimeric fusion proteins using a modification of the protocol of Seed and Aruffo (Proc. Natl. Acad. Sci. 84:3365 (1987)), incorporated by reference herein. Cells are seeded at 106 per 10 cm diameter culture dish 18-24 h before transfection. Plasmid DNA is added (approximately 15 μg/dish) in a volume of 5 mls of serum-free DMEM containing 0.1 mM chloroquine and 600 μg/ml DEAE Dextran, and cells are incubated for 3-3.5 h at 37° C. Transfected cells are then briefly treated (approximately 2 min) with 10% dimethyl sulfoxide in PBS and incubated at 37° C. for 16-24 h in DMEM containing 10% FCS. At 24 h after transfection, culture medium is removed and replaced with serum-free DMEM (6 ml/dish). Incubation is continued for 3 days at 37° C., at which time the spent medium is collected and fresh serum-free medium is added. After an additional 3 days at 37° C., the spent medium is again collected and cells are discarded. CHO cells expressing HBMYCNG-IgC-65 fusion proteins are isolated as described by Linsley et al., (1991) supra, as follows: stable transfectants expressing the desired fusion protein are isolated following cotransfection of dihydrofolate reductase-deficient Chinese hamster ovary (dhfr-CHO) cells with a mixture of the appropriate expression plasmid and the selectable marker, pSV2dhfr (Linsley et al., Proc. Natl. Acad. Sci. USA 87:5031 (1990)), incorporated by reference herein. Transfectants are then grown in increasing concentrations of methotrexate to a final level of 1 μM and were maintained in DMEM supplemented with 10% fetal bovine serum (FBS), 0.2 mM proline and 1 μM methotrexate. CHO lines expressing high levels of the desired fusion proteins are isolated by multiple rounds of fluorescence-activated cell sorting following indirect immunostaining with an appropriate labeled anti-HBMYCNG mAb.

Purification of Ig Fusion Proteins

The first, second and third collections of spent serum-free culture media from transfected COS cells are used as sources for the purification of Ig fusion proteins. After removal of cellular debris by low speed centrifugation, medium is applied to a column(approximately 200-400 ml medium/ml packed bed volume) of immobilized protein A (Repligen Corp., Cambridge, Mass.) equilibrated with 0.05 M sodium citrate, pH 8.0. After application of the medium, the column is washed with 1 M potassium phosphate, pH 8, and bound protein is eluted with 0.05 M sodium citrate, pH 3. Fractions were collected and immediately neutralized by addition of 1/10 volume of 2 M Tris, pH 8. Fractions containing the peak of A₂₈₀ absorbing material are pooled and dialyzed against PBS before use.

6.5 Preparation of Antibodies Directed Against HBMYCNG Epitopes

Antibodies of the present invention can be prepared by a variety of methods. In one method, purified HBMYCNG antigen or cells expressing purified HBMYCNG antigen are administered to an animal to induce the production of sera containing polyclonal antibodies. In a preferred method, a preparation of HBMYCNG antigen is purified to homogeneity before being administered to an animal to provide polyclonal antisera of greater specific activity. In certain embodiments, soluble portions of the HBMYCNG protein are used as the immunogen for generation of antibodies. Such soluble portions include, but are not limited to extracellular domains of the HBMYCNG protein which comprise, approximately residues 161 to 173, amino acid residues 237 to 274, and amino acid residues 370 to 453 of SEQ ID No. 2. In other embodiments, a soluble portion of the HBMYCNG protein used as an immunogen may include all or a portion of the amino terminal domain of the HBMYCNG protein, comprising, approximately, amino acid residues 1 or 2 to residue 140 SEQ ID No. 2, or all or a portion of the carboxy-terminal domain of the HBMYCNG protein, comprising, approximately amino acid residues 474 to 644 of SEQ ID No. 2. In other embodiments the immunogen administered to the animal may be a chimeric protein or peptide comprising a portion, particularly a soluble portion, of the HBMYCNG protein fused to a protein, polypeptide, or peptide carrier. Such fusions may be constructed by genetic engineering or may be formed by chemical conjugation of the HBMYCNG protein or peptide to a suitable carrier protein or peptide using methods well known in the art.

Monoclonal antibodies specific for the HBMYCNG protein, or a portion thereof, are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)).

An animal, preferably a mouse, is immunized with the HBMYCNG protein or a portion thereof and then splenocytes of the immunized mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981). Hybridoma cells obtained through such a selection are then assayed to identify clones which secrete antibodies capable of binding the HBMHCNG polypeptide or portion thereof.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art as disclosed above. (See also, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Isolation of Antibody Fragments Directed Against the HBMYCNG Protein from a Library of scFvs

Naturally occurring V-genes isolated from human peripheral blood lymphocytes (PBLs) are constructed into a library of antibody fragments which contain reactivities against the HBMYCNG protein to which the donor may or may not have been exposed (see e.g. Marks et al. J. Mol. Bio. 222(3): 581-97 (1991), and U.S. Pat. No. 5,885,793, each of which is incorporated herein by reference in its entirety).

A library of scFvs is constructed from the RNA of human PBLs as described in PCT publication WO 92/01047, which is hereby incorporated by reference in its entirety. To rescue phage displaying antibody fragments, approximately 10⁹ E. coli harboring the phagemid are used to inoculate 50 ml of 2× TY containing 1% glucose and 100 μg/ml of ampicillin (2× TY-AMP-GLU) and grown to an O.D. of 0.8 with shaking. Five ml of this culture are used to innoculate 50 ml of 2× TY-AMP-GLU, 2×10⁸ transforming units (TU) of M13 Δ gene III helper phage (PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2× TY containing 100 μg/ml ampicillin and 50 ug/ml kanamycin and grown overnight. Phage are prepared as described in PCT publication WO 92/01047.

M13 Δ gene III is prepared as follows: M13 Δ gene III helper phage does not encode gene III protein, hence the phage(mid) displaying antibody fragments have a greater avidity of binding to antigen. Infectious M13 Δ gene III particles are prepared by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are collected by centrifugation, resuspended in 300 ml 2× TY broth containing 100 μg ampicillin/ml and 25 μg kanamycin/ml (2× TY-AMP-KAN) and grown overnight, shaking at 37° C. Phage particles are purified and concentrated from the culture medium by two PEG-precipitations, resuspended in 2 ml PBS and passed through a 0.45 μm filter (Minisart NML; Sartorius) to give a final concentration of approximately 10¹³ transducing units/ml (ampicillin-resistant clones).

Panning of the Library. Immunotubes (Nunc) are coated overnight in PBS with 4 ml of either 100 μg/ml or 10 μg/ml of BMYCNG protein or portion thereof and then blocked with 2% Marvel-PBS for 2 hours at 37° C. and then washed 3 times in PBS. Approximately 10¹³ TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with A gene III helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Eluted phage from the 3rd and 4th rounds of selection are used to infect E. coli HB 2151 and soluble scfv is produced (Marks et al. J. Mol. Bio. 222(3): 581-97 (1991)) from single colonies for assay. ELISAs are performed with microtitre plates coated with either 10 pg/ml of HBMYCNG protein or a portion thereof in 50 mM bicarbonate pH 9.6. Clones positive in ELISA are further characterized by PCR fingerprinting (see, e.g., PCT publication WO 92/01047) and then by sequencing. 15

6.5 Method of Creating N- and C-terminal Deletion Mutants Corresponding to the HBMYCNG Polypeptide of the Present Invention.

As described elsewhere herein, the present invention encompasses the creation of N- and C-terminal deletion mutants, in addition to any combination of N- and C-terminal deletions thereof, corresponding to the HBMYCNG polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length HBMYCNG polypeptide sequence (as described herein, for example), appropriate primers of about 15-25 nucleotides derived from the desired 5′ and 3′ positions of SEQ ID NO:1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the Y140 to P664 N-terminal deletion mutant, the following primers could be used to amplify a cDNA fragment corresponding to this deletion mutant:

| | |
|---|---|
| 5′ Primer | (SEQ ID NO:19) |

5′-GCAGCA GCGGCCGC TACTACTGCTGGCTATTTGTCATTG-3′
NotI

| | |
|---|---|
| 3′ Primer | (SEQ ID NO:20) |

5′- GCAGCA GTCGAC TGGCTCGTCAGCAGCAGCCAGCTC-3′
Sal.I

For example, in the case of the M1 to F475 C-terminal deletion mutant, the following primers could be used to amplify a cDNA fragment corresponding to this deletion mutant:

| | |
|---|---|
| 5′ Primer | (SEQ ID NO:21) |
| 5′- GCAGCA GCGGCCGC ATGACCGAAAAAACCAATGGTGTG-3′ | |
| NotI | |
| 3′ Primer | (SEQ ID NO:22) |
| 5′- GCAGCA GTCGAC GAAGACCTGAGGACGGAGTTTCAG-3′ | |
| SalI | |

Representative PCR amplification conditions are provided below, although the skilled artisan would appreciate that other conditions may be required for efficient amplification. A 100 ul PCR reaction mixture may be prepared using 10 ng of the template DNA (cDNA clone of HBMYCNG), 200 uM 4dNTPs, 1 uM primers, 0.25U Taq DNA 5 polymerase (PE), and standard Taq DNA polymerase buffer. Typical PCR cycling condition are as follows:

20-25 cycles: 45 sec, 93 degrees

2 min, 50 degrees

2 min, 72 degrees

1 cycle: 10 min, 72 degrees

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). . The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula:

(S+(X*3)) to ((S+(X*3))+25), wherein ‘S’ is equal to the nucleotide position of the initiating start codon of the HBMYCNG gene (SEQ ID NO:1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25), wherein ‘S’ is equal to the nucleotide position of the initiating start codon of the HBMYCNG gene (SEQ ID NO:1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

In preferred embodiments, the following N-terminal HBMYCNG deletion polypeptides are encompassed by the present invention: M1-P664, T2-P664, E3-P664, K4-P664, T5-P664, N6-P664, G7-P664, V8-P664, K9-P664, S10-P664, S11-P664, P12-P664, A13-P664, N14-P664, N15-P664, H16-P664, N17-P664, H18-P664, H19-P664, A20-P664, P21-P664, P22-P664, A23-P664, 124-P664, K25-P664, A26-P664, N27-P664, G28-P664, K29-P664, D30-P664, D31-P664, H32-P664, R33-P664, T34-P664, S35-P664, S36-P664, R37-P664, P38-P664, H39-P664, S40-P664, A41-P664, A42-P664, D43-P664, D44-P664, D45-P664, T46-P664, S47-P664, S48-P664, E49-P664, L50-P664, Q51-P664, R52-P664, L53-P664, A54-P664, D55-P664, V56-P664, D57-P664, A58-P664, P59-P664, Q60-P664, Q61-P664, G62-P664, R63-P664, S64-P664, G65-P664, F66-P664, R67-P664, R68-P664, 169-P664, V70-P664, R71-P664, L72-P664, V73-P664, G74-P664, 175-P664, 176-P664, R77-P664, E78-P664, W79-P664, A80-P664, N81-P664, K82-P664, N83-P664, F84-P664, R85-P664, E86-P664, E87-P664, E88-P664, P89-P664, R90-P664, P91-P664, D92-P664, S93-P664, F94-P664, L95-P664, E96-P664, R97-P664, F98-P664, R99-P664, G100-P664, P101-P664, E102-P664, L103-P664, Q104-P664, T105-P664, V106-P664, T107-P664, T108-P664, Q109-P664, E110-P664, G111-P664, D112-P664, G113-P664, K114-P664, G115-P664, D116-P664, K117-P664, D118-P664, G119-P664, E120-P664, D121-P664, K122-P664, G123-P664, T124-P664, K125-P664, K126-P664, K127-P664, F128-P664, E129-P664, L130-P664, F131-P664, V132-P664, L133-P664, D134-P664, P135-P664, A136-P664, G137-P664, D138-P664, L139-P664, Y140-P664, Y141-P664, C142-P664, W143-P664, L144-P664, F145-P664, V146-P664, I147-P664, A148-P664, M149-P664, P150-P664, V151-P664, L152-P664, Y153-P664, N154-P664, W155-P664, C156-P664, L157-P664, L158-P664, V159-P664, A160-P664, R161-P664, A162-P664, C163-P664, F164-P664, S165-P664, D166-P664, L167-P664, Q168-P664, K169-P664, G170-P664, Y171-P664, Y172-P664, L173-P664, V174-P664, W175-P664, L176-P664, V177-P664, L178-P664, D179-P664, Y180-P664, V181-P664, S182-P664, D183-P664, V184-P664, V185-P664, Y186-P664, I187-P664, A188-P664, D189-P664, L190-P664, F191-P664, I192-P664, R193-P664, L194-P664, R195-P664, T196-P664, G197-P664, F198-P664, L199-P664, E200-P664, Q201-P664, G202-P664, L203-P664, L204-P664, V205-P664, K206-P664, D207-P664, T208-P664, K209-P664, K210-P664, L211-P664, R212-P664, D213-P664, N214-P664, Y215-P664, I216-P664, H217-P664, T218-P664, L219-P664, Q220-P664, F221-P664, K222-P664, L223-P664, D224-P664, V225-P664, A226-P664, S227-P664, I228-P664, I229-P664, P230-P664, T231-P664, D232-P664, L233-P664, I234-P664, Y235-P664, F236-P664, A237-P664, V238-P664, D239-P664, I240-P664, H241-P664, S242-P664, P243-P664, E244-P664, V245-P664, R246-P664, F247-P664, N248-P664, R249-P664, L250-P664, L251-P664, H252-P664, F253-P664, A254-P664, R255-P664, M256-P664, F257-P664, E258-P664, F259-P664, F260-P664, D261-P664, R262-P664, T263-P664, E264-P664, T265-P664, R266-P664, T267-P664, N268-P664, Y269-P664, P270-P664, N271-P664, I272-P664, F273-P664, R274-P664, I275-P664, S276-P664, N277-P664, L278-P664, V279-P664, L280-P664, Y281-P664, I282-P664, L283-P664, V284-P664, I285-P664, I286-P664, H287-P664, W288-P664, N289-P664, A290-P664, C291-P664, I292-P664, Y293-P664, Y294-P664, A295-P664, I296-P664, S297-P664, K298-P664, S299-P664, I300-P664, G301-P664, F302-P664, G303-P664, V304-P664, D305-P664, T306-P664, W307-P664, V308-P664, Y309-P664, P310-P664, N311-P664, I312-P664, T313-P664, D314-P664, P315-P664, E316-P664, Y317-P664, G318-P664, Y319-P664, L320-P664, A321-P664, R322-P664, E323-P664, Y324-P664, I325-P664, Y326-P664, C327-P664, L328-P664, Y329-P664, W330-P664, S331-P664, T332-P664, L333-P664, T334-P664, L335-P664, T336-P664, T337-P664, I338-P664, G339-P664, E340-P664, T341-P664, P342-P664, P343-P664, P344-P664, V345-P664, K346-P664, D347-P664, E348-P664, E349-P664, Y350-P664, L351-P664, F352-P664, V353-P664, I354-P664, F355-P664, D356-P664, F357-P664, L358-P664, I359-P664, G360-P664, V361-P664, L362-P664, I363-P664, F364-P664, A365-P664, T366-P664, I367-P664, V368-P664, G369-P664, N370-P664, V371-P664, G372-P664, S373-P664, M374-P664, I375-P664, S376-P664, N377-P664, M378-P664, N379-P664, A380-P664, T381-P664, R382-P664, A383-P664, E384-P664, F385-P664, Q386-P664, A387-P664, K388-P664, I389-P664, D390-P664, A391-P664, V392-P664, K393-P664, H394-P664, Y395-P664, M396-P664, Q397-P664, F398-P664, R399-P664, K400-P664, V401-P664, S402-P664, K403-P664, G404-P664, M405-P664, E406-P664, A407-P664, K408-P664, V409-P664, I410-P664, R411-P664, W412-P664, F413-P664, D414-P664, Y415-P664, L416-P664, W417-P664, T418-P664, N419-P664, K420-P664, K421-P664, T422-P664, V423-P664, D424-P664, E425-P664, R426-P664, E427-P664, I428-P664, L429-P664, K430-P664, N431-P664, L432-P664, P433-P664, A434-P664, K435-P664, L436-P664, R437-P664, A438-P664, E439-P664, I440-P664, A441-P664, T442-P664, N443-P664, V444-P664, H445-P664, L446-P664, S447-P664, T448-P664, L449-P664, K450-P664, K451-P664, V452-P664, R453-P664, I454-P664, F455-P664, H456-P664, D457-P664, C458-P664, E459-P664, A460-P664, G461-P664, L462-P664, L463-P664, V464-P664, E465-P664, L466-P664, V467-P664, L468-P664, K469-P664, L470-P664, R471-P664, P472-P664, Q473-P664, V474-P664, F475-P664, S476-P664, P477-P664, G478-P664, D479-P664, Y480-P664, I481-P664, C482-P664, R483-P664, K484-P664, G485-P664, D486-P664, I487-P664, G488-P664, K489-P664, E490-P664, M491-P664, Y492-P664, I493-P664, I494-P664, K495-P664, E496-P664, G497-P664, K498-P664, L499-P664, A500-P664, V501-P664, V502-P664, A503-P664, D504-P664, D505-P664, G506-P664, V507-P664, T508-P664, Q509-P664, Y510-P664, A511-P664, L512-P664, L513-P664, S514-P664, A515-P664, G516-P664, S517-P664, C518-P664, F519-P664, G520-P664, E521-P664, I522-P664, S523-P664, I524-P664, L525-P664, N526-P664, I527-P664, K528-P664, G529-P664, S530-P664, K531-P664, M532-P664, G533-P664, N534-P664, R535-P664, R536-P664, T537-P664, A538-P664, N539-P664, I540-P664, R541-P664, S542-P664, L543-P664, G544-P664, Y545-P664, S546-P664, D547-P664, L548-P664, F549-P664, C550-P664, L551-P664, S552-P664, K553-P664, D554-P664, D555-P664, L556-P664, M557-P664, E558-P664, A559-P664, V560-P664, T561-P664, E562-P664, Y563-P664, P564-P664, D565-P664, A566-P664, K567-P664, K568-P664, V569-P664, L570-P664, E571-P664, E572-P664, R573-P664, G574-P664, R575-P664, E576-P664, I577-P664, L578-P664, M579-P664, K580-P664, E581-P664, G582-P664, L583-P664, L584-P664, D585-P664, E586-P664, N587-P664, E588-P664, V589-P664, A590-P664, T591-P664, S592-P664, M593-P664, E594-P664, V595-P664, D596-P664, V597-P664, Q598-P664, E599-P664, K600-P664, L601-P664, G602-P664, Q603-P664, L604-P664, E605-P664, T606-P664, N607-P664, M608-P664, E609-P664, T610-P664, L611-P664, Y612-P664, T613-P664, R614-P664, F615-P664, G616-P664, R617-P664, L618-P664, L619-P664, A620-P664, E621-P664, Y622-P664, T623-P664, G624-P664, A625-P664, Q626-P664, Q627-P664, K628-P664, L629-P664, K630-P664, Q631-P664, R632-P664, I633-P664, T634-P664, V635-P664, L636-P664, E637-P664, T638-P664, K639-P664, M640-P664, K641-P664, Q642-P664, N643-P664, N644-P664, E645-P664, D646-P664, D647-P664, Y648-P664, L649-P664, S650-P664, D651-P664, G652-P664, M653-P664, N654-P664, S655-P664, P656-P664, E657-P664, and/or L658-P664 of SEQ ID NO:2. Polynucleotide sequences encoding these polypeptides are also provided. The present invention also encompasses the use of these N-terminal HBMYCNG deletion polypeptides as immunogenic and/or antigenic epitopes as described elsewhere herein.

In preferred embodiments, the following C-terminal HBMYCNG deletion polypeptides are encompassed by the present invention: M1-P664, M1-E663, M1-D662, M1-A661, M1-A660, M1-A659, M1-L658, M1-E657, M1-P656, M1-S655, M1-N654, M1-M653, M1-G652, M1-D651, M1-S650, M1-L649, M1-Y648, M1-D647, M1-D646, M1-E645, M1-N644, M1-N643, M1-Q642, M1-K641, M1-M640, M1-K639, M1-T638, M1-E637, M1-L636, M1-V635, M1-T634, M1-I633, M1-R632, M1-Q631, M1-K630, M1-L629, M1-K628, M1-Q627, M1-Q626, M1-A625, M1-G624, M1-T623, M1-Y622, M1-E621, M1-A620, M1-L619, M1-L618, M1-R617, M1-G616, M1-F615, M1-R614, M1-T613, M1-Y612, M1-L611, M1-T610, M1-E609, M1-M608, M1-N607, M1-T606, M1-E605, M1-L604, M1-Q603, M1-G602, M1-L601, M1-K600, M1-E599, M1-Q598, M1-V597, M1-D596, M1-V595, M1-E594, M1-M593, M1-S592, M1-T591, M1-A590, M1-V589, M1-E588, M1-N587, M1-E586, M1-D585, M1-L584, M1-L583, M1-G582, M1-E581, M1-K580, M1-M579, M1-L578, M1-I577, M1-E576, M1-R575, M1-G574, M1-R573, M1-E572, M1-E571, M1-L570, M1-V569, M1-K568, M1-K567, M1-A566, M1-D565, M1-P564, M1-Y563, M1-E562, M1-T561, M1-V560, M1-A559, M1-E558, M1-M557, M1-L556, M1-D555, M1-D554, M1-K553, M1-S552, M1-L551, M1-C550, M1-F549, M1-L548, M1-D547, M1-S546, M1-Y545, M1-G544, M1-L543, M1-S542, M1-R541, M1-I540, M1-N539, M1-A538, M1-T537, M1-R536, M1-R535, M1-N534, M1-G533, M1-M532, M1-K531, M1-S530, M1-G529, M1-K528, M1-I527, M1-N526, M1-L525, M1-I524, M1-S523, M1-I522, M1-E521, M1-G520, M1-F519, M1-C518, M1-S517, M1-G516, M1-A515, M1-S514, M1-L513, M1-L512, M1-A511, M1-Y510, M1-Q509, M1-T508, M1-V507, M1-G506, M1-D505, M1-D504, M1-A503, M1-V502, M1-V501, M1-A500, M1-L499, M1-K498, M1-G497, M1-E496, M1-K495, M1-I494, M1-I493, M1-Y492, M1-M491, M1-E490, M1-K489, M1-G488, M1-I487, M1-D486, M1-G485, M1-K484, M1-R483, M1-C482, M1-I481, M1-Y480, M1-D479, M1-G478, M1-P477, M1-S476, M1-F475, M1-V474, M1-Q473, M1-P472, M1-R471, M1-L470, M1-K469, M1-L468, M1-V467, M1-L466, M1-E465, M1-V464, M1-L463, M1-L462, M1-G461, M1-A460, M1-E459, M1-C458, M1-D457, M1-H456, M1-F455, M1-I454, M1-R453, M1-V452, M1-K451, M1-K450, M1-L449, M1-T448, M1-S447, M1-L446, M1-H445, M1-V444, M1-N443, M1-T442, M1-A441, M1-I440, M1-E439, M1-A438, M1-R437, M1-L436, M1-K435, M1-A434, M1-P433, M1-L432, M1-N431, M1-K430, M1-L429, M1-I428, M1-E427, M1-R426, M1-E425, M1-D424, M1-V423, M1-T422, M1-K421, M1-K420,

M1-N419, M1-T418, M1-W417, M1-L416, M1-Y415, M1-D414, M1-F413, M1-W412, M1-R411, M1-I410, M1-V409, M1-K408, M1-A407, M1-E406, M1-M405, M1-G404, M1-K403, M1-S402, M1-V401, M1-K400, M1-R399, M1-F398, M1-Q397, M1-M396, M1-Y395, M1-H394, M1-K393, M1-V392, M1-A391, M1-D390, M1-I389, M1-K388, M1-A387, M1-Q386, M1-F385, M1-E384, M1-A383, M1-R382, M1-T381, M1-A380, M1-N379, M1-M378, M1-N377, M1-S376, M1-I375, M1-M374, M1-S373, M1-G372, M1-V371, M1-N370, M1-G369, M1-V368, M1-I367, M1-T366, M1-A365, M1-F364, M1-I363, M1-L362, M1-V361, M1-G360, M1-I359, M1-L358, M1-F357, M1-D356, M1-F355, M1-I354, M1-V353, M1-F352, M1-L351, M1-Y350, M1-E349, M1-E348, M1-D347, M1-K346, M1-V345, M1-P344, M1-P343, M1-P342, M1-T341, M1-E340, M1-G339, M1-I338, M1-T337, M1-T336, M1-L335, M1-T334, M1-L333, M1-T332, M1-S331, M1-W330, M1-Y329, M1-L328, M1-C327, M1-Y326, M1-I325, M1-Y324, M1-E323, M1-R322, M1-A321, M1-L320, M1-Y319, M1-G318, M1-Y317, M1-E316, M1-P315, M1-D314, M1-T313, M1-I312, M1-N311, M1-P310, M1-Y309, M1-V308, M1-W307, M1-T306, M1-D305, M1-V304, M1-G303, M1-F302, M1-G301, M1-I300, M1-S299, M1-K298, M1-S297, M1-1296, M1-A295, M1-Y294, M1-Y293, M1-I292, M1-C291, M1-A290, M1-N289, M1-W288, M1-H287, M1-I286, M1-I285, M1-V284, M1-L283, M1-I282, M1-Y281, M1-L280, M1-V279, M1-L278, M1-N277, M1-S276, M1-I275, M1-R274, M1-F273, M1-I272, M1-N271, M1-P270, M1-Y269, M1-N268, M1-T267, M1-R266, M1-T265, M1-E264, M1-T263, M1-R262, M1-D261, M1-F260, M1-F259, M1-E258, M1-F257, M1-M256, M1-R255, M1-A254, M1-F253, M1-H252, M1-L251, M1-L250, M1-R249, M1-N248, M1-F247, M1-R246, M1-V245, M1-E244, M1-P243, M1-S242, M1-H241, M1-I240, M1-D239, M1-V238, M1-A237, M1-F236, M1-Y235, M1-I234, M1-L233, M1-D232, M1-T231, M1-P230, M1-I229, M1-I228, M1-S227, M1-A226, M1-V225, M1-D224, M1-L223, M1-K222, M1-F221, M1-Q220, M1-L219, M1-T218, M1-H217, M1-I216, M1-Y215, M1-N214, M1-D213, M1-R212, M1-L211 M-K210,, M1-K209, M1-T208, M1-D207, M1-K206, M1-V205, M1-L204, M1-L203, M1-G202, M1-Q201, M1-E200, M1-L199, M1-F198, M1-G197, M1-T196, M1-R195, M1-L194, M1-R193, M1-1192, M1-F191, M1-L190, M1-D189, M1-A188, M1-I187, M1-Y186, M1-V185, M1-V184, M1-D183, M1-S182, M1-V181, M1-Y180, M1-D179, M1-L178, M1-V177, M1-L176, M1-W175, M1-V174, M1-L173, M1-Y172, M1-Y171, M1-G170, M1-K169, M1-Q168, M1-L167, M1-D166, M1-S165, M1-F164, M1-C163, M1-A162, M1-R161, M1-A160, M1-V159, M1-L158, M1-L157, M1-C156, M1-W155, M1-N154, M1-Y153, M1-L152, M1-V151, M1-P150, M1-M149, M1-A148, M1-I147, M1-V146, M1-F145, M1-L144, M1-W143, M1-C142, M1-Y141, M1-Y140, M1-L139, M1-D138, M1-G137, M1-A136, M1-P135, M1-D134, M1-L133, M1-V132, M1-F131, M1-L130, M1-E129, M1-F128, M1-K127, M1-K126, M1-K125, M1-T124, M1-G123, M1-K122, M1-D121, M1-E120, M1-G119, M1-D118, M1-K117, M1-D116, M1-G115, M1-K114, M1-G113, M1-D112, M1-G111, M1-E110, M1-Q109, M1-T108, M1-T107, M1-V106, M1-T105, M1-Q104, M1-L103, M1-E102, M1-P101, M1-G100, M1-R99, M1-F98, M1-R97, M1-E96, M1-L95, M1-F94, M1-S93, M1-D92, M1-P91, M1-R90, M1-P89, M1-E88, M1-E87, M1-E86, M1-R85, M1-F84, M1-N83, M1-K82, M1-N81, M1-A80, M1-W79, M1-E78, M1-R77, M1-176, M1-175, M1-G74, M1-V73, M1-L72, M1-R71, M1-V70, M1-169, M1-R68, M1-R67, M1-F66, M1-G65, M1-S64, M1-R63, M1-G62, M1-Q61, M1-Q60, M1-P59, M1-A58, M1-D57, M1-V56, M1-D55, M1-A54, M1-L53, M1-R52, M1-Q51, M1-L50, M1-E49, M1-S48, M1-S47, M1-T46, M1-D45, M1-D44, M1-D43, M1-A42, M1-A41, M1-S40, M1-H39, M1-P38, M1-R37, M1-S36, M1-S35, M1-T34, M1-R33, M1-H32, M1-D31, M1-D30, M1-K29, M1-G28, M1-N27, M1-A26, M1-K25, M1-124, M1-A23, M1-P22, M1-P21, M1-A20, M1-H19, M1-H18, M1-N17, M1-H16, M1-N15, M1-N14, M1-A13, M1-P12, M1-S11, M1-S10, M1-K9, M1-V8, and/or M1-G7 of SEQ ID NO:2. Polynucleotide sequences encoding these polypeptides are also provided. The present invention also encompasses the use of these C-terminal HBMYCNG deletion polypeptides as immunogenic and/or antigenic epitopes as described elsewhere herein.

The present invention also encompasses the the same N- and/or C-terminal deletion mutants for the varant HBMYCNG polypeptide depicted in FIG. 6 (SEQ ID NO:24) with the appropriate amino acid and encoding nucleic acid substitutions. Methods of substituting such sequences are known in the art.

6.6 Method of Enhancing the Biological Activity/Functional Characteristics of Invention Through Molecular Evolution.

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered ion channel protein may be constitutively active upon binding of its cognate ligand. Alternatively, an engineered ion channel protein may be constitutively active in the absence of ligand binding. In yet another example, an engineered ion channel protein may be capable of being activated with less than all of the regulatory factors and/or conditions typically required for ion channel protein activation (e.g., ion flux, ligand binding, phosphorylation, conformational changes, etc.). Such ion channel protein would be useful in screens to identify ion channel protein modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as descibed by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny. DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments -further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 bp fragments could be eluted from said paper using 1M NaCL, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for 5 min using an MJ Research (Cambridge, Mass.) PTC-150 thermocycler. After the assembly reaction is completed, a 1:40 dilution of the resulting primeness product would then be introduced into a PCR mixture (using the same buffer mixture used for the assembly reaction) containing 0.8 um of each primer and subjecting this mixture to 15 cycles of PCR (using 94 C for 30s, 50 C for 30s, and 72 C for 30s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailered to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host, particularly if the polynucleotides and polypeptides provide a therapeutic use. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. The forgoing are hereby incorporated in their entirety herein for all purposes.

### 7. DEPOSIT OF MICROORGANISMS

The following microorganisms were deposited with the American Type Culture Collection (ATCC), 10801 University Blvd., Manassas, Va. 20110 on ______ and assigned the following numbers:

Microorganism ATCC Deposit No. HBMYCNG-pcDNA _______

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various publications are cited herein, the disclosures of which are incorporated by reference in their entireties.

We claim:

1. An isolated nucleic acid molecule comprising a member of the group consisting of:
(a) a nucleotide sequence that encodes a polypeptide having the amino acid sequence of FIG. 2;
(b) the complement of the nucleotide sequence of (a);
(c) a HBMYCNG gene or a complement of a HBMYCNG gene as contained in ATCC Deposit No. ______;
(d) an isolated nucleic acid molecule comprising nucleotides 23 to 2011 of SEQ ID NO:1, wherein said nucleotides encode a polypeptide of SEQ ID NO:2 minus the start codon;
(e) an isolated nucleic acid molecule comprising nucleotides 20 to 2011 of SEQ ID NO:1, wherein said nucleotides encode a polypeptide of SEQ ID NO:2 including the start codon;
(f) An isolated nucleic acid molecule comprising the nucleotide sequence of FIG. 1;
(g) A nucleic acid molecule comprising a nucleotide sequence encoding a deletion mutant of HBMYCNG or the complement of the nucleotide sequence of the deletion mutant of HBMYCNG;
(h) a nucleic acid molecule capable of hybridizing to and which is at least 95% identical to a nucleic acid molecule of (a), (b), (c), (d), (e), (f), or (g); and
(i) An isolated nucleic acid molecule of (h), further comprising a label.

2. An isolated nucleic acid molecule comprising a nucleotide sequence that hybridizes to the nucleic acid of claim 1 and encodes a naturally occurring HBMYCNG polypeptide.

3. An isolated nucleic acid molecule of claim 2 further comprising the nucleotide sequence linked uninterrupted by stop codons to a nucleotide sequence that encodes a heterologous protein or peptide.

4. A recombinant vector containing the nucleotide sequence of claim 1.

5. A genetically engineered host cell containing the nucleotide sequence of claim 1.

6. The genetically engineered host cell of claim 5 containing the nucleotide sequence of claim 1 operatively associated with a regulatory nucleotide sequence containing transcriptional and translational regulatory information that controls expression of the nucleotide sequence in a host cell.

7. A method of making an HBMYCNG polypeptide comprising the steps of:
(a) culturing the cell of claim 6 in an appropriate culture medium to produce an HBMYCNG polypeptide; and
(b) isolating the HBMYCNG polypeptide.

8. The method of claim 7, wherein the HBMYCNG polypeptide is HBMYCNG or a functionally equivalent derivative thereof.

9. An antibody preparation which is specifically reactive with an epitope of an HBMYCNG polypeptide.

10. A transgenic animal comprising the nucleic acid molecule of claim 1.

11. A substantially pure polypeptide comprising a member of the group selected from:
(a) A substantially pure polypeptide encoded by the nucleic acid molecule of claim 1;
(b) A substantially pure human polypeptide, as depicted in FIG. 2;
(c) A substantially pure polypeptide which is at least 95% identical to the polypeptide as set forth in FIG. 2;
(d) A substantially pure polypeptide comprising amino acids 2 to 664 of SEQ ID NO:2, wherein said amino acids 2 to 664 comprise a polypeptide of SEQ ID NO:2 minus the start methionine; and
(e) A substantially pure polypeptide comprising amino acids 1 to 664 of SEQ ID NO:2.

12. A fusion protein comprising a polypeptide of claim 11 and a second heterologous polypeptide.

13. A test kit for detecting and/or quantitating a wild type or mutant HBMYCNG nucleic acid molecule in a sample, comprising the steps of contacting the sample with a nucleic acid molecule of claim 1; and detecting and/or quantitating the label as an indication of the presence or absence and/or amount of a wild type or mutant HBMYCNG nucleic acid.

14. A test kit for detecting and/or quantitating a wild type or mutant HBMYCNG polypeptide in a sample, comprising the steps of contacting the sample with the antibody of claim 9; and detecting and/or quantitating a polypeptide-antibody complex as an indication of the presence or absence and/or amount of a wild type or mutant HBMYCNG nucleic acid.

15. A method for identifying compounds that modulate HBMYCNG activity comprising:
(a) contacting a test compound to a cell that expresses a HBMYCNG gene;
(b) measuring the level of HBMYCNG gene expression in the cell; and
(c) comparing the level obtained in (b) with the HBMYCNG gene expression obtained in the absence of the compound;
such that if the level obtained in (b) differs from that obtained in the absence of the compound, a compound that modulates HBMYCNG activity is identified.

16. A method for identifying compounds that modulate HBMYCNG activity comprising:
(a) contacting a test compound to a cell that contains a HBMYCNG polypeptide;
(b) measuring the level of HBMYCNG polypeptide or activity in the cell; and
(c) comparing the level obtained in (b) with the level of HBMYCNG polypeptide or activity obtained in the absence of the compound;
such that if the level obtained in (b) differs from that obtained in the absence of the compound, a compound that modulates HBMYCNG activity is identified.

17. A method for identifying compounds that regulate ion channel-related disorders, comprising:
(a) contacting a test compound with a cell which expresses a nucleic acid of claim 1, and
(b) determining whether the test compound modulates HBMYCNG activity.

18. A method for the treatment of ion channel-related disorders, comprising administering an effective amount of a compound that increases expression of a HBMYCNG gene.

19. A pharmaceutical formulation for the treatment of ion channel-related disorders, comprising a compound that activates or inhibits HBMYCNG activity, mixed with a pharmaceutically acceptable carrier.

20. A method for identifying compounds that modulate the activity of an ion channel comprising:
(a) contacting a test compound to a cell that expresses a HBMYCNG gene and the ion channel, and measuring Ca+2 flux into the cell;
(b) contacting a test compound to a cell that expresses a HBMYCNG gene but does not express the ion channel, and measuring Ca+2 flux into the cell; and
(c) comparing Ca+2 flux obtained in (b) with the Ca+2 flux obtained in (a);
such that if the level obtained in (b) differs from that obtained in (b), a compound that modulates ion channel activity is identified.

21. An isolated nucleic acid molecule consisting of a member of the group consisting of:
(a) a nucleotide sequence that encodes a polypeptide having the amino acid sequence of FIG. 2;
(b) the complement of the nucleotide sequence of (a);
(c) a HBMYCNG gene or a complement of a HBMYCNG gene as contained in ATCC Deposit No. ______;
(d) an isolated nucleic acid molecule comprising nucleotides 23 to 2011 of SEQ ID NO:1, wherein said nucleotides encode a polypeptide of SEQ ID NO:2 minus the start codon;
(e) an isolated nucleic acid molecule comprising nucleotides 20 to 2011 of SEQ ID NO:1, wherein said nucleotides encode a polypeptide of SEQ ID NO:2 including the start codon;
(f) An isolated nucleic acid molecule comprising the nucleotide sequence of FIG. 1;
(g) A nucleic acid molecule comprising a nucleotide sequence encoding a deletion mutant of HBMYCNG or the complement of the nucleotide sequence of the deletion mutant of HBMYCNG;
(h) a nucleic acid molecule capable of hybridizing to and which is at least 95% identical to a nucleic acid molecule of (a), (b), (c), (d), (e), (f), or (g); and
(i) An isolated nucleic acid molecule of (h), further comprising a label.

22. A substantially pure polypeptide consisting of a member of the group selected from:
(a) A substantially pure polypeptide encoded by the nucleic acid molecule of claim 1;
(b) A substantially pure human polypeptide, as depicted in FIG. 2;
(c) A substantially pure polypeptide which is at least 95% identical to the polypeptide as set forth in FIG. 2;
(d) A substantially pure polypeptide comprising amino acids 2 to 664 of SEQ ID NO:2, wherein said amino acids 2 to 664 comprise a polypeptide of SEQ ID NO:2 minus the start methionine; and
(e) A substantially pure polypeptide comprising amino acids 1 to 664 of SEQ ID NO:2.
